(19) United States
(12) Patent Application Publication
(10) Pub. No.: US 2015/0045289 A1
(43) Pub. Date: Feb. 12, 2015

(54) METHOD OF PRE-PREPARING MEDICATIONS FOR THERAPEUTIC USES

(71) Applicant: Alchemia Oncology Pty Limited, Eight Mile Plains (AU)

(72) Inventors: MICHAEL L. WEST, Birkdale (AU); Tracey J. BROWN, Flemington (AU)

(21) Appl. No.: 14/330,512

(22) Filed: Jul. 14, 2014

Related U.S. Application Data
(60) Provisional application No. 61/846,507, filed on Jul. 15, 2013.

(51) Int. Cl. A61J 1/10; A61K 31/4745; B65B 3/00; A61K 31/7068; A61J 1/20; A61K 31/513; A61K 38/12

(52) U.S. Cl. CPC A61J 1/10; A61K 31/513; A61K 31/4745; A61K 38/12; A61K 31/7068; A61J 1/2089; B65B 3/003
USPC 514/2.9; 514/274; 514/283; 514/49; 604/246; 53/473

(57) ABSTRACT

The present invention relates to the method of pre-preparing pharmaceutical compositions for therapeutic uses, particularly pre-preparing medications for treating patients (e.g., cancer patients and the like) by intravenous infusion.

### CROSS REFERENCE TO RELATED APPLICATIONS

This application claims priority to U.S. Provisional Application Ser. No. 61/846,507, filed Jul. 15, 2013, and titled “Method of Pre-Preparing Medications for Therapeutic Uses,” which is hereby incorporated by reference in its entirety.

### FIELD OF THE INVENTION

The present invention relates to the method of pre-preparing pharmaceutical compositions for therapeutic uses, particularly pre-preparing medications for treating cancer patients and the like by intravenous infusion.

### INTRODUCTION

The preparation, handling, and administration of intravenous drugs can be challenging. Processes and safeguards necessary for safe and efficient handling of intravenous (IV) medications depend on several factors. Highly qualified and disciplined staff are required for dispensing medications. This process may involve a pharmacist or nurse dispensing, diluting, formulating or otherwise compounding one or more pharmaceutical drugs, diluents and/or delivery agents according to a specific protocol under aseptic conditions.

The use of body-surface area (BSA) has been the mainstay of chemotherapy (CHT) dosing in oncology practice for the last half-century. Derived from animal models to estimate appropriate phase I drug doses, BSA equates to the two-dimensional surface area of a person's skin (Field, et al., Journal of Oncology Practice 4(3): 108-113 (2008)). Hence, optimal chemotherapy dosing, established through clinical trials, is generally calculated using a patient's BSA, which takes into account body weight and height. Consequently, in the dosing of most chemotherapeutic agents, every patient has an individualized dosing requirement based on their BSA.

In the case of IV antibiotics and other drugs, dose adjustment based on patient actual weight or ideal weight is often used. Other methods of patient-specific dosing are known to the medical practitioner.

Prior to any treatment, the doctor may prescribe the name of the drug, the dose to be administered, the dates of treatment, the method of administration, the type of diluent or carrier in which the drug is to be diluted and administered, and the duration of the treatment.

The next stage is generally for a pharmacist or a nurse to prepare the medication based on the dosage prescribed by the doctor. The standard procedure for preparing medication for intravenous infusion is to compound the components under aseptic conditions prior to administration to the patient. This compounding may take place within the hospital pharmacy or at an outsourced central compounding facility, and is typically done not more than 48 hours prior to administration because of the need to maintain sterility of the formulated dose. For example, in a typical preparation of chemotherapy, based on BSA, the prescribed dosage of the cytotoxic drug is withdrawn from one or more vials and mixed with an amount of solvent or diluent(s) (such as 5% dextrose, 0.9% saline, Ringer's solution, etc.). The amount of solvent or diluent(s) is typically controlled by available package sizes, such as 100, 200, 500 and 1000 mL bags or bottles. This method of drug preparation allows the dose to be altered from patient to patient since the active agent or drug is withdrawn from separate vial(s) and added to the solvent. However, because the volume of solvent is constant, the concentration of active agent or drug varies depending on the patient's BSA.

The use of fixed volumes of solvents within a hospital pharmacy significantly reduces waste, reduces the number of manipulations of the dosage and thereby reduces the potential for the sterility to be compromised. Using an entire package of solvent also increases efficiency within the busy pharmacy. A limited number of pack sizes for typical solvents also increases the efficiency of the back office ordering, stock control and storage systems within the hospital.

Currently used alternatives to the standard hospital protocol for intravenous delivery of therapeutics include use of pre-filled multi-chamber bags such as the DUPLEX® Drug Delivery System developed by BBraun. This system has two or more chambers in one bag. The thin membrane between the chambers is broken just before administration. This allows a concentrate in one chamber to mix with a solvent in the other chamber or a drug in one chamber to mix with a drug in the other chamber. This type of bag is suitable when the drug is not stable in dilute solution or the mixture is not stable after mixing. Multi-chamber bags represent a move towards pre-prepared/packaged drug products which have improved control of the quality and quantity of medication within the device. The disadvantage with multi-chamber bags, however, is that they are not predisposed to dosage adjustment. The amount of drug in an isolated chamber is fixed for all patients. Consequently, multi-chamber bags are not suitable for the administration of drugs or therapeutic agents which must be dosed on some patient-specific parameter such as BSA, body weight, or any other measurable parameter.

Eliuk et al. (U.S. Pat. No. 8,386,070) have developed an automated pharmacy system for preparing intermediary IV bags as drug sources for creating highly diluted patient doses in syringes. The automated pharmacy system is a robotic system which is preloaded with sources of various drugs, solvents and IV bags. Once programmed, the system will reconstitute, withdraw, dilute and mix one or more drugs into an IV bag which serves as a reservoir for the withdrawal of the formulated mixture into syringes ready for administration. The system replaces the manual tasks normally undertaken by the hospital pharmacist in preparing an IV dosage for a patient. As with the standard hospital compounding protocol, the automated system is designed to prepare dosages just prior to administration to the patient.

Standard methods of preparing IV dosages, whether through manual compounding or some form of automated pharmacy system, may be constrained by a number of factors. The first is the need to prepare the final formulation by diluting the drug(s) in the appropriate solvent(s) and or delivery agent(s) shortly before administration to the patient. This need may arise because the pharmacopeia mandates a limited storage time for medicaments prepared within this environment based on sterility considerations. A direct consequence of both the short interval between preparation and use, and the fact that each dosage is potentially different, may be that there is no formal quality control testing for dosage forms prepared in this manner. Multi-chamber bag systems and other pre-mix bag systems are amenable to much more rigorous quality control testing. However these systems do not allow for dosage adjustment; they are designed to provide the same dose to all patients.

When weight adjusted dosages of drugs are prepared under the standard procedures, whether by manual manipulation or by the use of an automated system, the concentration of the active agent varies from patient to patient, with larger patients receiving a more concentrated infusate than smaller patients. Chemotherapeutic drugs in particular may cause local toxic effects at the infusion site, ranging from erythema, phlebitis, urticaria and pain to severe tissue necrosis when extravasated (Brande & Vermorken, CME Journal of Gynecologic Oncology 6(1): 43-51 (2001)). The extent to which these side effects occur has been linked to the concentration of the active agent in the infusion solution (Yamada et al., Free Radical Biology & Medicine 48(1): 120-27 (2010); Clinical biochemistry 1998; Hamilton, http://www.gics.com.au/resources/OncEmergencies_Extravasation_KH.pdf).

Consequently there is a need for a patient dosing system that allows thorough quality control testing, provides for extended shelf life of the formulated dosage, allows the dosage to be adjusted from patient to patient as required and allows for a fixed concentration of active agent within the infusion solution.

It is the object of the present invention to overcome or substantially ameliorate at least one of the above problems or disadvantages.

### SUMMARY OF INVENTION

Provided herein are methods for intravenous delivery of a patient-adjusted dose of a therapeutic agent in a solution of predetermined concentration to a patient in need thereof, comprising administering intravenously to the patient in need thereof a dosage volume of the solution having a predetermined concentration of the therapeutic agent, wherein the predetermined concentration of the therapeutic agent is used for each patient treated with the therapeutic agent at a particular dosage strength, and wherein the predetermined concentration of the therapeutic agent is independent of the patient-adjusted dose, and wherein the dosage volume is selected to deliver the patient-adjusted dose to the patient.

Also provided herein are uses of a patient-adjusted dose of a therapeutic agent in a solution of predetermined concentration for a patient in need thereof for intravenous delivery, comprising use of a dosage volume of the solution having a predetermined concentration of the therapeutic agent, wherein the predetermined concentration of the therapeutic agent is used for each patient treated with the therapeutic agent at a particular dosage strength, and wherein the predetermined concentration of the therapeutic agent is independent of the patient-adjusted dose, and wherein the dosage volume is selected to deliver the patient-adjusted dose to the patient.

In some embodiments, the patient-adjusted dose of the therapeutic agent is a function of at least one patient characteristic. In some embodiments, the patient characteristic is selected from the group consisting of the patient's body surface area, the patient's body weight or ideal body weight, the patient's hepatic function, and the patient's renal function. In some embodiments, the patient characteristic is the patient's body surface area. In some embodiments, the patient characteristic is patient's body weight or ideal body weight. In some embodiments, the patient characteristic is the patient's hepatic function. In some embodiments, the patient characteristic is the patient's renal function.

In some embodiments, the therapeutic agent is photolabile. In some embodiments, the therapeutic agent is selected from the group consisting of an antibiotic, an antiviral, a muscle relaxant, a sedative, an anesthetic, a cancer chemotherapeutic agent, and an antifungal. In some embodiments the therapeutic agent is a cancer chemotherapeutic agent (for example, a small molecule chemotherapeutic agent). In some embodiments, the therapeutic agent is selected from the group consisting of irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine. In some embodiments, the therapeutic agent is a glucocorticoid. In some embodiments, the therapeutic agent is selected from the group consisting of dexamethasone and cortisol. In some embodiments, the therapeutic agent comprises irinotecan. In some embodiments, the therapeutic agent comprises semisynthetic irinotecan. In some embodiments, the therapeutic agent comprises synthetic irinotecan.

In some embodiments, the predetermined concentration of the therapeutic agent in the solution is a therapeutically effective concentration. In some embodiments, the therapeutic agent comprises irinotecan, wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL. In some embodiments, the therapeutic agent comprises irinotecan, wherein the predetermined concentration is 0.72 mg/mL. In some embodiments, the therapeutic agent comprises irinotecan, wherein the predetermined concentration is 0.6-0.8 mg/mL. In some embodiments, the therapeutic agent comprises irinotecan, wherein the predetermined concentration is 0.6 mg/mL. In some embodiments, the therapeutic agent comprises irinotecan, wherein the predetermined concentration is 0.5-0.7 mg/mL. In some embodiments, the therapeutic agent comprises irinotecan, wherein the predetermined concentration is 0.48 mg/mL. In some embodiments, the therapeutic agent comprises irinotecan, wherein the predetermined concentration is 0.4-0.6 mg/mL.

In some embodiments, the solution of predetermined concentration of the therapeutic agent further comprises one or more excipients or adjuvants. In some embodiments, the one or more excipients or adjuvants are selected from the group consisting of sodium hyaluronate, lactic acid, sorbitol, and glucose. In some embodiments the solution includes an adjuvant and the adjuvant is sodium hyaluronate (for example, the sodium hyaluronate described herein). In some embodiments the therapeutic agent is a cancer chemotherapeutic agent (for example, a small molecule chemotherapeutic agent) and an adjuvant is present and is sodium hyaluronate. In some embodiments, the solution of predetermined concentration of the therapeutic agent further comprises a diluent, wherein the diluent is selected from the group consisting of saline, Ringer's solution, and glucose solution. In some embodiments, the diluent comprises five percent glucose solution.

In some embodiments, the patient is a mammal. In some embodiments, the mammal is selected from the group consisting of bovine, canine, equine, feline, porcine, and human. In some embodiments the mammal is human.

Additionally, provided herein are systems for intravenous delivery of a patient-adjusted dose of a therapeutic agent in a solution of predetermined concentration to a patient in need thereof, comprising at least one pharmacy container containing the solution of predetermined concentration of the therapeutic agent, wherein the at least one pharmacy container is configured to allow transfer of multiple portions of the solution; one or more patient dosing containers each containing an initial volume of the solution of predetermined concentration of the therapeutic agent, wherein the one or more patient dosing containers are configured to allow volume adjustment and are configured for intravenous administration of the solution to the patient, wherein each of the one or more patient dosing containers may contain the same or different initial volumes of the solution, and wherein the predetermined concentration of the solution in the at least one pharmacy container and the one or more patient dosing containers is the same concentration.

In some embodiments, the volume adjustment comprises introduction of a top-up volume of the solution from the at least one pharmacy container into one or more of the one or more patient dosing containers. In some embodiments, the volume adjustment comprises withdrawal of a surplus volume of the solution from one or more of the patient dosing containers. In some embodiments, the volume adjustment comprises introduction of a top-up volume of the solution from the at least one pharmacy container into at least one patient dosing container, and withdrawal of a surplus volume of the solution from at least one other patient dosing container.

In some embodiments, the system further comprises instructions for preparing the patient-adjusted dose of the therapeutic agent, wherein the instructions indicate that the patient-adjusted dose of the therapeutic agent can be prepared using a specified volume of the solution of predetermined concentration. In some embodiments, the instructions further describe the specified volume of the solution of predetermined concentration required to provide the patient-adjusted dose of the therapeutic agent as a function of at least one patient characteristic. In some embodiments, the patient characteristic is selected from the group consisting of the patient's body surface area, the patient's body weight or ideal body weight, the patient's hepatic function, and the patient's renal function. In some embodiments, the patient characteristic is the patient's body surface area. In some embodiments, the patient characteristic is patient's body weight or ideal body weight. In some embodiments, the patient characteristic is the patient's hepatic function. In some embodiments, the patient characteristic is the patient's renal function.

In some embodiments, the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume. In some embodiments, the system further comprises instructions for preparing the patient adjusted dose, wherein the instructions describe the number of patient dosing containers from the first group and the number of patient dosing containers from the second group and the volume, if any, of the solution of predetermined concentration from the pharmacy container required to provide the patient-adjusted dose. In some embodiments, the first volume is 200 mL and the second volume is 300 mL. In some embodiments, the first volume is 150 mL and the second volume is 250 mL. In some embodiments, the first volume is 150 mL and the second volume is 200 mL. In some embodiments, the pharmacy container contains 200 mL of the solution of predetermined concentration. In some embodiments, the one or more patient dosing containers further comprise a third group of one or more patient dosing containers, wherein the third group of one or more patient dosing containers contains a third volume of the solution of predetermined concentration, and wherein the third volume is different from the second volume. In some embodiments, the one or more patient dosing containers further comprise a fourth group of one or more patient dosing containers, wherein the fourth group of one or more patient dosing containers contains a fourth volume of the solution of predetermined concentration, and wherein the fourth volume is different from the third volume. In some embodiments, the first volume is 60 mL, the second volume is 100 mL, the third volume is 200 mL, and the fourth volume is 300 mL. In some embodiments, the pharmacy container contains 50 mL of the solution of predetermined concentration. In some embodiments, the pharmacy container contains 20 mL of the solution of predetermined concentration. In some embodiments, each of the one or more patient dosing containers in the first group and in the second group has a maximum capacity, and wherein the maximum capacity of the one or more patient dosing containers in the first group is 300 mL and the maximum capacity of the one or more patient dosing containers in the second group is 400 mL. In some embodiments, each of the one or more patient dosing containers in the first group and in the second group has a maximum capacity, and wherein the maximum capacity of the one or more patient dosing containers in the first group is 250 mL and the maximum capacity of the one or more patient dosing containers in the second group is 300 mL.

In some embodiments, the one or more patient dosing containers comprise one or more materials, wherein the one or more materials are selected from the group consisting of plastic and glass. In some embodiments, the at least one pharmacy container comprises one or more materials, wherein the one or more materials are selected from the group consistent of plastic and glass. In some embodiments, the one or more materials comprise polypropylene. In some embodiments, the one or more patient dosing containers comprise triple layer polypropylene bags. In some embodiments, the at least one pharmacy container comprises a triple layer polypropylene bag. In some embodiments, the at least one pharmacy container comprises a syringe. In some embodiments, the one or more patient dosing containers limit light transmission to the solution. In some embodiments, the one or more patient dosing containers comprise on or more materials that reflect light. In some embodiments, the one or more patient dosing containers comprise a foil layer. In some embodiments, the one or more patient dosing containers comprise a double-laminated foil package. In some embodiments, the one or more patient dosing containers comprise one or more materials that absorb light. In some embodiments, the at least one pharmacy container limits light transmission to the solution. In some embodiments, the at least one pharmacy container limits light transmission to the solution. In some embodiments, the at least one pharmacy container reflects light. In some embodiments, the at least one pharmacy container comprises a foil layer. In some embodiments, the at least one pharmacy container comprises a double-laminated foil package. In some embodiments, the at least one pharmacy container comprises one or more materials that absorb light.

In some embodiments, the system comprises at least two patient dosing containers, further comprising connecting tubes, wherein the connecting tubes connect the patient dosing containers. In some embodiments, the connecting tubes comprise a small bore Y-extension set.

In some embodiments, the therapeutic agent is photolabile. In some embodiments, the therapeutic agent is selected from the group consisting of an antibiotic, an antiviral, a muscle relaxant, a sedative, an anesthetic, a cancer chemotherapeutic agent, and an antifungal. In some embodiments the therapeutic agent is a cancer chemotherapeutic agent (for example, a small molecule chemotherapeutic agent). In some embodiments, the therapeutic agent is selected from the group consisting of irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine. In some embodiments, the therapeutic agent is a glucocorticoid. In some embodiments, the therapeutic agent is selected from the group consisting of dexamethasone and cortisol. In some embodiments, the therapeutic agent comprises irinotecan. In some embodiments, the therapeutic agent comprises semisynthetic irinotecan. In some embodiments, the therapeutic agent comprises synthetic irinotecan.

In some embodiments, the predetermined concentration of the therapeutic agent in the solution is a therapeutically effective concentration. In some embodiments, the therapeutic agent comprises irinotecan, wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL. In some embodiments, the therapeutic agent comprises irinotecan, wherein the predetermined concentration is 0.72 mg/mL. In some embodiments, the therapeutic agent comprises irinotecan, wherein the predetermined concentration is 0.6-0.8 mg/mL. In some embodiments, the therapeutic agent comprises irinotecan, wherein the predetermined concentration is 0.6 mg/mL. In some embodiments, the therapeutic agent comprises irinotecan, wherein the predetermined concentration is 0.5-0.7 mg/mL. In some embodiments, the therapeutic agent comprises irinotecan, wherein the predetermined concentration is 0.48 mg/mL. In some embodiments, the therapeutic agent comprises irinotecan, wherein the predetermined concentration is 0.4-0.6 mg/mL.

In some embodiments, the solution of predetermined concentration of the therapeutic agent further comprises one or more excipients or adjuvants. In some embodiments, the one or more excipients or adjuvants are selected from the group consisting of sodium hyaluronate, lactic acid, sorbitol, and glucose. In some embodiments the solution includes an adjuvant and the adjuvant is sodium hyaluronate (for example, the sodium hyaluronate described herein). In some embodiments the therapeutic agent is a cancer chemotherapeutic agent (for example, a small molecule chemotherapeutic agent) and an adjuvant is present and is sodium hyaluronate. In some embodiments, the solution of predetermined concentration of the therapeutic agent further comprises a diluent, wherein the diluent is selected from the group consisting of saline, Ringer's solution, and glucose solution. In some embodiments, the diluent comprises five percent glucose solution.

In some embodiments, the patient is a mammal. In some embodiments, the mammal is selected from the group consisting of bovine, canine, equine, feline, porcine, and human. In some embodiments the mammal is human.

Additionally, provided herein is a method of preparing a patient-adjusted dose of a therapeutic agent for intravenous delivery to a patient in need thereof using a system provided herein, comprising determining a dosage volume of the solution of predetermined concentration of the therapeutic agent to be administered to the patient, wherein the dosage volume is the volume of solution required to provide the patient-adjusted dose; and selecting one or more patient dosing containers containing the solution of predetermined concentration of the therapeutic agent, wherein if the total volume in the one or more patient dosing containers is less than the dosage volume, then adding a top-up volume of the solution of predetermined concentration of the therapeutic agent from a pharmacy container to one or more of the one or more patient dosing containers, such that after addition of the top-up volume, the total volume of solution in the one or more patient dosing containers is equal to the dosage volume, wherein the predetermined concentration of the solution in the pharmacy container and the one or more patient dosing containers is the same concentration, and wherein the pharmacy container is configured to allow transfer of multiple portions of the solution, or if the total volume in the one or more patient dosing containers is more than the dosage volume, then withdrawing a surplus volume of the solution of predetermined concentration from one or more of the one or more patient dosing containers, such that after withdrawal of the surplus volume, the total volume of solution in the one or more patient dosing containers is equal to the dosage volume, wherein the one or more patient dosing containers are configured to allow volume adjustment and are configured for intravenous administration of the solution to the patient.

In some embodiments, the total volume in the one or more patient dosing containers is less than the dosage volume. In some embodiments, the volume adjustment comprises introduction of a top-up volume of the solution from the at least one pharmacy container into one or more of the one or more patient dosing containers. In some embodiments, the total volume in the one or more patient dosing containers is less than the dosage volume. In some embodiments, the volume adjustment comprises withdrawal of a surplus volume of the solution from one or more of the patient dosing containers. In some embodiments, the volume adjustment comprises introduction of a top-up volume of the solution from the at least one pharmacy container into at least one patient dosing container, and withdrawal of a surplus volume of the solution from at least one other patient dosing container.

In some embodiments, the patient-adjusted dose of the therapeutic agent is a function of at least one patient characteristic. In some embodiments, the patient characteristic selected from the group consisting of the patient's body surface area, the patient's body weight or ideal body weight, the patient's hepatic function, and the patient's renal function. In some embodiments, the patient characteristic is the patient's body surface area. In some embodiments, the patient characteristic is patient's body weight or ideal body weight. In some embodiments, the patient characteristic is the patient's hepatic function. In some embodiments, the patient characteristic is the patient's renal function.

In some embodiments, the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume.

In some embodiments, the one or more patient dosing containers comprise one or more materials, wherein the one or more materials are selected from the group consisting of plastic and glass. In some embodiments, the at least one pharmacy container comprises one or more materials, wherein the one or more materials are selected from the group consistent of plastic and glass. In some embodiments, the one or more materials comprise polypropylene. In some embodiments, the one or more patient dosing containers comprise triple layer polypropylene bags. In some embodiments, the at least one pharmacy container comprises a triple layer polypropylene bag. In some embodiments, the at least one pharmacy container comprises a syringe. In some embodiments, the one or more patient dosing containers limit light transmission to the solution. In some embodiments, the one or more patient dosing containers comprise on or more materials that reflect light. In some embodiments, the one or more patient dosing containers comprise a foil layer. In some embodiments, the one or more patient dosing containers comprise a double-laminated foil package. In some embodiments, the one or more patient dosing containers comprise one or more materials that absorb light. In some embodiments, the at least one pharmacy container limits light transmission to the solution. In some embodiments, the at least one pharmacy container limits light transmission to the solution. In some embodiments, the at least one pharmacy container reflects light. In some embodiments, the at least one pharmacy container comprises a foil layer. In some embodiments, the at least one pharmacy container comprises a double-laminated foil package. In some embodiments, the at least one pharmacy container comprises one or more materials that absorb light.

In some embodiments, the therapeutic agent is photolabile. In some embodiments, the therapeutic agent is selected from the group consisting of an antibiotic, an antiviral, a muscle relaxant, a sedative, an anesthetic, a cancer chemotherapeutic agent, and an antifungal. In some embodiments the therapeutic agent is a cancer chemotherapeutic agent (for example, a small molecule chemotherapeutic agent). In some embodiments, the therapeutic agent is selected from the group consisting of irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine. In some embodiments, the therapeutic agent is a glucocorticoid. In some embodiments, the therapeutic agent is selected from the group consisting of dexamethasone and cortisol. In some embodiments, the therapeutic agent comprises irinotecan. In some embodiments, the therapeutic agent comprises semisynthetic irinotecan. In some embodiments, the therapeutic agent comprises synthetic irinotecan.

In some embodiments, the predetermined concentration of the therapeutic agent in the solution is a therapeutically effective concentration. In some embodiments, the therapeutic agent comprises irinotecan, wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL. In some embodiments, the therapeutic agent comprises irinotecan, wherein the predetermined concentration is 0.72 mg/mL. In some embodiments, the therapeutic agent comprises irinotecan, wherein the predetermined concentration is 0.6-0.8 mg/mL. In some embodiments, the therapeutic agent comprises irinotecan, wherein the predetermined concentration is 0.6 mg/mL. In some embodiments, the therapeutic agent comprises irinotecan, wherein the predetermined concentration is 0.5-0.7 mg/mL. In some embodiments, the therapeutic agent comprises irinotecan, wherein the predetermined concentration is 0.48 mg/mL. In some embodiments, the therapeutic agent comprises irinotecan, wherein the predetermined concentration is 0.4-0.6 mg/mL.

In some embodiments, the solution of predetermined concentration of the therapeutic agent further comprises one or more excipients or adjuvants. In some embodiments, the one or more excipients or adjuvants are selected from the group consisting of sodium hyaluronate, lactic acid, sorbitol, and glucose. In some embodiments the solution includes an adjuvant and the adjuvant is sodium hyaluronate (for example, the sodium hyaluronate described herein). In some embodiments the therapeutic agent is a cancer chemotherapeutic agent (for example, a small molecule chemotherapeutic agent) and an adjuvant is present and is sodium hyaluronate. In some embodiments, the solution of predetermined concentration of the therapeutic agent further comprises a diluent, wherein the diluent is selected from the group consisting of saline, Ringer's solution, and glucose solution. In some embodiments, the diluent comprises five percent glucose solution.

In some embodiments, the patient is a mammal. In some embodiments, the mammal is selected from the group consisting of bovine, canine, equine, feline, porcine, and human. In some embodiments the mammal is human.

Additionally, provided herein are methods of manufacturing a system described herein for intravenous delivery of a patient-adjusted dose of a therapeutic agent in a solution of predetermined concentration to a patient in need thereof, comprising: formulating a solution for intravenous delivery, wherein the solution comprises a predetermined concentration of the therapeutic agent; and packaging the solution in a plurality of containers, wherein the plurality of containers comprise one or more patient dosing containers and at least one pharmacy container, wherein the one or more patient dosing containers are configured to allow volume adjustment and are configured for intravenous administration of the solution to a patient, and wherein the at least one pharmacy container is configured to allow transfer of multiple portions of the solution.

In some embodiments, the volume adjustment comprises introduction of a top-up volume of the solution from the at least one pharmacy container into one or more of the one or more patient dosing containers. In some embodiments, the volume adjustment comprises withdrawal of a surplus volume of the solution from one or more of the patient dosing containers. In some embodiments, the volume adjustment comprises introduction of a top-up volume of the solution from the at least one pharmacy container into at least one patient dosing container, and withdrawal of a surplus volume of the solution from at least one other patient dosing container.

In some embodiments, the patient-adjusted dose of the therapeutic agent is a function of at least one patient characteristic. In some embodiments, the patient characteristic is selected from the group consisting of the patient's body surface area, the patient's body weight or ideal body weight, the patient's hepatic function, and the patient's renal function. In some embodiments, the patient characteristic is the patient's body surface area. In some embodiments, the patient characteristic is patient's body weight or ideal body weight. In some embodiments, the patient characteristic is the patient's hepatic function. In some embodiments, the patient characteristic is the patient's renal function.

In some embodiments, the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume.

In some embodiments, the one or more patient dosing containers comprise one or more materials, wherein the one or more materials are selected from the group consisting of plastic and glass. In some embodiments, the at least one pharmacy container comprises one or more materials, wherein the one or more materials are selected from the group consistent of plastic and glass. In some embodiments, the one or more materials comprise polypropylene. In some embodiments, the one or more patient dosing containers comprise triple layer polypropylene bags. In some embodiments, the at least one pharmacy container comprises a triple layer polypropylene bag. In some embodiments, the at least one pharmacy container comprises a syringe. In some embodiments, the one or more patient dosing containers limit light transmission to the solution. In some embodiments, the one or more patient dosing containers comprise on or more materials that reflect light. In some embodiments, the one or more patient dosing containers comprise a foil layer. In some embodiments, the one or more patient dosing containers comprise a double-laminated foil package. In some embodiments, the one or more patient dosing containers comprise one or more materials that absorb light. In some embodiments, the at least one pharmacy container limits light transmission to the solution. In some embodiments, the at least one pharmacy container limits light transmission to the solution. In some embodiments, the at least one pharmacy container reflects light. In some embodiments, the at least one pharmacy container comprises a foil layer. In some embodiments, the at least one pharmacy container comprises a double-laminated foil package. In some embodiments, the at least one pharmacy container comprises one or more materials that absorb light.

In some embodiments, the therapeutic agent is photolabile. In some embodiments, the therapeutic agent is selected from the group consisting of an antibiotic, an antiviral, a muscle relaxant, a sedative, an anesthetic, a cancer chemotherapeutic agent, and an antifungal. In some embodiments the therapeutic agent is a cancer chemotherapeutic agent (for example, a small molecule chemotherapeutic agent). In some embodiments, the therapeutic agent is selected from the group consisting of irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine. In some embodiments, the therapeutic agent is a glucocorticoid. In some embodiments, the therapeutic agent is selected from the group consisting of dexamethasone and cortisol. In some embodiments, the therapeutic agent comprises irinotecan. In some embodiments, the therapeutic agent comprises semisynthetic irinotecan. In some embodiments, the therapeutic agent comprises synthetic irinotecan.

In some embodiments, the predetermined concentration of the therapeutic agent in the solution is a therapeutically effective concentration. In some embodiments, the therapeutic agent comprises irinotecan, wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL. In some embodiments, the therapeutic agent comprises irinotecan, wherein the predetermined concentration is 0.72 mg/mL. In some embodiments, the therapeutic agent comprises irinotecan, wherein the predetermined concentration is 0.6-0.8 mg/mL. In some embodiments, the therapeutic agent comprises irinotecan, wherein the predetermined concentration is 0.6 mg/mL. In some embodiments, the therapeutic agent comprises irinotecan, wherein the predetermined concentration is 0.5-0.7 mg/mL. In some embodiments, the therapeutic agent comprises irinotecan, wherein the predetermined concentration is 0.48 mg/mL. In some embodiments, the therapeutic agent comprises irinotecan, wherein the predetermined concentration is 0.4-0.6 mg/mL.

In some embodiments, the solution of predetermined concentration of the therapeutic agent further comprises one or more excipients or adjuvants. In some embodiments, the one or more excipients or adjuvants are selected from the group consisting of sodium hyaluronate, lactic acid, sorbitol, and glucose. In some embodiments the solution includes an adjuvant and the adjuvant is sodium hyaluronate (for example, the sodium hyaluronate described herein). In some embodiments the therapeutic agent is a cancer chemotherapeutic agent (for example, a small molecule chemotherapeutic agent) and an adjuvant is present and is sodium hyaluronate. In some embodiments, the solution of predetermined concentration of the therapeutic agent further comprises a diluent, wherein the diluent is selected from the group consisting of saline, Ringer's solution, and glucose solution. In some embodiments, the diluent comprises five percent glucose solution.

In some embodiments, the patient is a mammal. In some embodiments, the mammal is selected from the group consisting of bovine, canine, equine, feline, porcine, and human. In some embodiments the mammal is human.

In some embodiments, packaging the solution in the plurality of containers comprises aseptic filtration of the solution.

Additionally, provided herein are methods for intravenous delivery of a patient-adjusted dose of a therapeutic agent in a solution of predetermined concentration to a patient in need thereof, using one of the systems described herein.

In certain embodiments are provided uses of the systems described herein for the intravenous delivery of a patient-adjusted dose of a therapeutic agent in a solution of predetermined concentration to a patient in need thereof.

In certain embodiments are provided uses of a therapeutic agent in the preparation of a medicament for treating a patient in need thereof, wherein the therapeutic agent is for intravenous administration in a solution having a predetermined concentration of the agent.

In certain embodiments are provided uses of a therapeutic agent in the preparation of a medicament for treating a patient in need thereof, wherein the agent is for intravenous administration in a solution and wherein a dose is selected for the patient by adjusting the volume of the solution to be administered without adjusting the concentration of the agent therein.

In certain embodiments are provided uses of a therapeutic agent in the preparation of a medicament for treating a patient in need thereof, wherein the therapeutic agent is for intravenous administration in a solution of predetermined concentration, wherein the predetermined concentration of the therapeutic agent is for use in each patient treated with the therapeutic agent at a particular dosage strength, and wherein the dose is patient-adjusted by adjusting the volume of the solution to be administered.

In certain embodiments are provided uses of a therapeutic agent in the preparation of a medicament for treating a patient in need thereof, wherein the therapeutic agent is for intravenous delivery in a dosage volume of a solution having a predetermined concentration of the therapeutic agent, wherein the predetermined concentration of the therapeutic agent is for use in each patient treated with the therapeutic agent at a particular dosage strength, and wherein the predetermined concentration of the therapeutic agent is independent of the patient-adjusted dose, and wherein the dosage volume is selected to deliver the patient-adjusted dose to the patient.

Additionally, provided herein are therapeutic agents for use in treating a patient in need thereof wherein the therapeutic agent is for intravenous administration in a solution having a predetermined concentration of the agent.

Additionally, provided herein are therapeutic agents for use in treating a patient in need thereof wherein the therapeutic agent is for intravenous administration in a solution and wherein the dose is selected for the patient by adjusting the volume of the solution to be administered without adjusting the concentration of the agent therein.

Additionally, provided herein are therapeutic agents for use in treating a patient in need thereof wherein the therapeutic agent is for intravenous administration in a solution of predetermined concentration, wherein the predetermined concentration of the therapeutic agent is for use in each patient treated with the therapeutic agent at a particular dosage strength, and wherein the dose is patient-adjusted by adjusting the volume of the solution to be administered.

Additionally, provided herein are therapeutic agents for use in treating a patient in need thereof wherein the therapeutic agent is for intravenous delivery in a dosage volume of a solution having a predetermined concentration of the therapeutic agent, wherein the predetermined concentration of the therapeutic agent is for use in each patient treated with the therapeutic agent at a particular dosage strength, and wherein the predetermined concentration of the therapeutic agent is independent of the patient-adjusted dose, and wherein the dosage volume is selected to deliver the patient-adjusted dose to the patient.

### BRIEF DESCRIPTION OF THE DRAWINGS

FIG. 1 shows an example of a first patient dosing container (10) and a second patient dosing container (60) disposed in a parallel configuration. Each of the first and second patient dosing containers (10) and (60) in this example is fitted with a first port (20) to facilitate the addition or withdrawal of formulation. The first port (20) may be a conventional septum device designed to be pierced by a syringe needle, or a needle free valve suitable for the purpose. The first and second patient dosing containers (10) and (60) are each further equipped with a second port (30) designed for connecting with a “giving set” (40). Each giving set (40) is connected to a piggy-back device (70), which may optionally contain a one way valve device. The piggy-back device (70) is further connected to the patient (50), either directly by a further giving set, or by means of an infusion pump device.

FIG. 2 shows an example of a first patient dosing container (110) and a second patient dosing container (160) connected in series such that the formulation from the second patient dosing container (160) flows into first patient dosing container (110) via connecting tube (140). Each of the first and second dosing containers (110) and (160) in this example is fitted with a first port (120) to facilitate the addition or withdrawal of formulation. The first port (120) may be a conventional septum device designed to be pierced by a syringe needle, or a needle free valve suitable for the purpose. The first and second patient dosing containers (110) and (160) are each further equipped with a second port (130) designed for allowing the efflux of formulation. A patient connecting tube (170) attached to patient dosing container (110) via second port (130) is further connected to an infusion pump device or directly to the patient (150).

### DETAILED DESCRIPTION

The present invention relates to the method of pre-preparing medications for therapeutic uses, particularly pre-preparing medications for intravenous use. Particularly, the concentration of the active agent, for example, a chemotherapeutic drug, may be pre-prepared at a constant concentration in a container supplied by the manufacturer. The container may be sterile and subject to quality control in order to ensure the sterility and quality of the formulation therein according to regulatory standards.

The therapeutic agents may include, but are not limited to, cytotoxic agents, antibiotic agents and other agents commonly used for treating patients by intravenous (IV) infusion, including but not limited to irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, azathiprine, bleomycin, bortezomib, busulfan, capecitabine, chlorambucil, cytarabine, liposomal doxorubicin, etoposide, etoposide phosphate, fludarabine, fotemustine, ganciclovir, hydroxyurea, lomustine, melphalan, pemetrexed, raltitrexed, temozolomide, thiotepa, valganciclovir. daunomycin, dactinomycin, esorubicin, mafosfamide, cytosine arabinoside, bis-chloroethyInitrosurea, actinomycin D, mithramycin, prednisone, colchicine, hydroxyprogesterone, testosterone, tamoxifen, procarbazine, hexamethylmelamine, pentamethylmelamine, amsacrine, methylcyclohexyInitrosurea, nitrogen mustards, 6-mercaptopurine, 6-thioguanine, 5-azacytidine, deoxycoformycin, 4-hydroxyperoxycyclophosphoramide, 5-fluorodeoxyuridine (5-FUdR), methotrexate (MTX), colchicine, taxol, trimetrexate, topotecan, and diethylstilbestrol (DES). Therapeutic agents may include, but are not limited to, small molecule chemical agents, biological agents such as proteins, peptides and nucleic acids, as well as antibodies. For example, the antibody, fragment, derivative or portion thereof may be selected from the group of antibodies consisting of: ABX-EGF, Alemtuzumab, Apolizumab, Bevacizumab (avastin), Cantuzumab, Cetuximab, cG250, cmc-544, Daclizumab, Epratuzumab, erlotinib, Gemtuzumab ozogamicin, hA20; HCBE-I 1, Hun901, Ibritumomab tiuxetan, IDEC 159, Infliximab, Lumiliximab, mAb 3F8, mAb b43.13, mAb BC8, mAb CC49-deltaCH2, mAb Ch14.18, mAb CP-675,206, mAb HeFi-I, mAb Hu3S193, mAb HuGl-M195, mAb huHMFG1, mAb J591, mAb MDX-CTLA4, mAb MiK-beta-1, MDX-010, MEDI-507, MLN2704, Pertuzumab, RAV12, Rituximab, SGN-30, SGN-40, Tositumomab, Trastuzumab (herceptin), TRM-I (TRAIL Rl Mab), and Yttrium-ibritumomab. In some embodiments the therapeutic agent is a cancer chemotherapeutic agent. In some embodiments the cancer chemotherapeutic agent is a small molecule. In some embodiments, the chemotherapeutic agent is a protein. In some embodiments, therapeutic agent is an antibody.

Further, the pharmaceutical composition of the present invention may comprise any pharmaceutically acceptable additive, carrier, and/or adjuvant or excipient that may promote the transfer of this agent via intravenous infusion.

Intravenous infusion solutions and diluents may include sterile water, dextrose, saline, lactated Ringer's solution, and the like.

As used herein, a “pharmaceutical carrier” is a pharmaceutically acceptable solvent or delivery agent, suspending agent or vehicle for delivering the therapeutic agent to the animal or human. Such a carrier is known and conventionally used in the art to facilitate the storage, administration, and/or the biological activity of therapeutic agent(s) within a pharmaceutical composition of the present invention. A carrier may also reduce any undesirable side effects of the components of such a pharmaceutical composition. A suitable carrier should be stable, i.e., it should not react with other ingredients in the formulation. It should not produce significant local or systemic adverse effect in recipients at the dosages and concentrations employed for treatment.

Suitable carriers for the various embodiments of the present invention may include those conventionally used for large stable macromolecules such as but not limited to albumin, gelatin, collagen, polysaccharide such as hyaluronic acid, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), Tween 80, microspheres, nanoparticles and the like. Amongst various carriers utilized for target-oriented drug delivery, vesicular drug delivery systems in the form of liposome, microsomes and niosomes have been most extensively investigated.

The usefulness of sodium hyaluronate (also known as hyaluronan or hyaluronic acid (HA)) as an adjuvant for chemotherapeutics when co-administered with these drugs has been reported in International Patent Application Nos. PCT/AU00/00004 (International Pub. No. WO 00/041730) and PCT/AU01/00849 (International Pub. No. WO 02/005852), U.S. patent application Ser. No. 11/191,407 (U.S. Pub. No. 2005/0267069 and granted as U.S. Pat. No. 8,287,894), and U.S. Ser. No. 11/198,663 (U.S. Pub. No. 2006/0178342), which are incorporated in their entirety herein by reference. Sodium hyaluronate is a naturally occurring polysaccharide comprising linear-chain polymers, which is found ubiquitously throughout the animal kingdom. Sodium hyaluronate with an intrinsic viscosity of between 6.0 and 18.5 dl/gm is suitable for use in intravenous drug delivery, with sodium hyaluronate of intrinsic viscosity between 10.0 and 14.5 dl/g particularly suitable for use as an adjuvant in combination with chemotherapeutic agents. As will be appreciated by the skill artisan, intrinsic viscosity is a property of biopolymers that can be described by the Mark-Houwink equation (see en.wikipedia.org/wiki/Mark % E2%80%93Houwink_equation) and that can be measured empirically. For example, see the European Pharmacopeia entry for sodium hyaluronate monograph 1472 (2010), which outlines the standard test methods and tolerances. In addition to the methods outlined in the European Pharmacopeia, there are a number of commercially available instruments for the automated determination of intrinsic viscosity. Instructions and methods appropriate for each instrument are provided by the manufacturers.

Dosage strength defines the amount of active component or substance (e.g., pharmaceutical drug) per unit volume. The quantitative composition in terms of active substance represents the strength. The concept of strength and the concept of concentration are linked. The strength represents the amount of active substance in the pharmaceutical form, which can be defined as a concentration. For preparations containing a single active agent the strength is defined as the amount of active substance per unit volume (e.g., mg/mL). Drugs are often prescribed to be delivered at a particular total exposure (e.g., mg/kg, mg/m²) due to factors such as the patient's general health. In certain circumstances there may be more than one therapeutic agent present. Treatments in which there is more than one therapeutic agent present may require adjustment of the dose of one agent independently of the other. Under these circumstances, changing the dosage volume will not necessarily provide a satisfactory result because this will change the dose of both therapeutic agents in the formulation. In order to allow the delivery of different doses of active substance (e.g., where formulated to deliver different mg/m² or mg/kg as prescribed), and also to allow the administration of particular dosages of more than one active substance independent of each other, the formulation may be presented in several dosage strengths to allow the adjustment of the dose of one agent independently of the other. Example 3 described herein presents an exemplary embodiment of such a system in which there are formulations at three dosage strengths of irinotecan but where the strength of sodium hyaluronate is constant: 0.72 mg/mL irinotecan and 4 mg/mL sodium hyaluronate; 0.60 mg/mL irinotecan and 4 mg/mL sodium hyaluronate; and 0.48 mg/mL irinotecan and 4 mg/mL sodium hyaluronate.

The active component or substance of the present invention is administered to a subject, such as a mammal, or a patient, in a pharmaceutically acceptable form and in a therapeutically effective concentration. A composition is said to be “pharmacologically acceptable” if its administration can be tolerated by a recipient patient. Such an agent is said to be administered in a “therapeutically effective amount” if the amount administered is physiologically significant. An agent is physiologically significant if its presence results in a detectable change in the physiology of a recipient patient. In some embodiments in which the therapeutic agent comprises irinotecan, semi-synthetic irinotecan, or synthetic irinotecan, the USP monograph for irinotecan does not distinguish between semi-synthetic and synthetic irinotecan except in relation to the impurities and tests thereof. In some embodiments in which the therapeutic agent comprises irinotecan, semi-synthetic irinotecan, or synthetic irinotecan, the predetermined concentration may be 0.72 mg/mL ±5%. In other embodiments in which the therapeutic agent comprises irinotecan, semi-synthetic irinotecan, or synthetic irinotecan, the predetermined concentration may be 0.6 mg/mL ±5%. In other embodiments in which the therapeutic agent comprises irinotecan, semi-synthetic irinotecan, or synthetic irinotecan, the predetermined concentration may be 0.48 mg/mL ±5%. In some embodiments in which the therapeutic agent comprises irinotecan, semi-synthetic irinotecan, or synthetic irinotecan, the predetermined concentration may be within the range 0.12 mg/mL ±5% to 2.8 mg/mL ±5%. In some embodiments, the predetermined concentration of the therapeutic agent may be less than 0.1 mg/mL and greater than 0 mg/mL. In some embodiments, the predetermined concentration of the therapeutic agent may be from 0.1 mg/mL to 1 mg/mL, 0.1 mg/mL to 0.8 mg/mL, 0.1 mg/mL to 0.7 mg/mL, 0.1 mg/mL to 0.6 mg/mL, 0.1 mg/mL to 0.5 mg/mL, 0.1 mg/mL to 0.4 mg/mL, 0.4 mg/mL to 1 mg/mL, 0.4 mg/ml to 0.8 mg/mL, or 0.1 mg/mL to 0.6 mg/mL. In some embodiments the predetermined concentration of the therapeutic agent may be from 1 μg/mL to 1000 μg/mL, 1 μg/mL to 500 μg/mL, 1 μg/mL to 250 μg/mL, 1 μg/mL to 100 μg/mL, 1 to 50 μg/mL, 1 to 25 μg/mL, or 1 to 10 μg/mL. In some embodiments the predetermined concentration of the therapeutic agent may be from 10 μg/mL to 1000 μg/mL, 10 μg/mL to 500 μg/mL, 10 μg/mL to 250 μg/mL, 10 μg/mL to 100 μg/mL, 10 to 50 μg/mL, or 10 to 25 μg/mL. In some embodiments the predetermined concentration of the therapeutic agent may be from 100 μg/mL to 1000 μg/mL, 100 μg/mL to 500 μg/mL, 100 μg/mL to 250 μg/mL, or 100 μg/mL to 300 μg/mL. In other embodiments, the predetermined concentration of the therapeutic agent may be 1 mg/mL to 10 mg/mL. In some embodiments, the predetermined concentration of the therapeutic agent may be 5 mg/mL to 15 mg/mL. In some embodiments, the predetermined concentration of the therapeutic agent may be 10 mg/mL to 20 mg/mL. In some embodiments, the predetermined concentration of the therapeutic agent may be 15 mg/mL to 25 mg/mL. Based on the teachings provided herein and the information available to the skilled artisan for dosing protocols for known therapeutic agents, the skilled artisan will also be able to select appropriate predetermined concentrations based on the dosing needs of patients and approved dosing ranges known in the field. For recited ranges of predetermined concentrations or volumes (e.g. top-off or surplus volumes) as recited herein, as is appreciated by skilled artisans, for recited values it is intended that there is a tolerance of ±5%-±10%. In some embodiments, the tolerance is ±5%. The particular dosage for a specific agent will be apparent to the skilled addressee. For known therapeutic agents, guidance can be found in the prescribing information published by the manufacturer of the drug product. Certain agents may be delivered at dosages outside the dosage recommended by the manufacturer, particularly in the treatment of cancer. Alternative dosages are often published in the scientific literature and guidance can be found for example in the National Comprehensive Cancer Network (NCCN) guidelines (http://www.nccn.org/professionals/physician_gls/f_guidelines. asp) and the National Institute for Health and Care Excellence Guidelines (NICE) (http://www.nice.org.uk/). For new therapeutic agents, guidance on dosage is derived from animal studies and clinical trials for the agent. The instant system is amenable to use over an extensive range of dosages based on the patient's illness, ailment, condition, side effects and overall reaction to the treatment and dosage. The dosage may be varied depending on a number of factors including the age of the patient, BSA, ethnic group and whether or not any other ailments exist.

The instant system is amenable to bulk sterilization processes such as terminal sterilization, as well as pre-sterilization of components by means such as gamma radiation or autoclaving, followed by aseptic fill. The choice of sterilization methods for the system will depend on the susceptibility of the constituent ingredients to each of the alternative methods available to the manufacturer.

Sterile injectable solutions can be prepared by incorporating the active compound (e.g., antibiotic, chemotherapeutic agent) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above.

The pharmaceutical preparation for IV infusion can be enclosed in bags or other suitable containers, such as syringes, made of glass or plastic, for example, the patient dosing containers described herein.

Patient-adjusted dose refers to the dose of an agent based on a specific patient characteristic. Many therapeutic agents are given at a standard dose to the majority of adult patients of normal liver function. For example, acetaminophen (paracetemol) may be given at a dose of 1 gm per patient every 4-6 hours. Dosages of this form are not considered to be patient-adjusted dosages because they do not take into account individual patient characteristics. In contrast, the dose of many treatments for cancer and a number of other conditions are based on physical or chemical characteristics of the individual to be treated. Such dosing is considered to be patient-adjusted dosing. Characteristics which are often used to determine patient-adjusted dosing include BSA, body weight, ideal body weight, body mass index (BMI), systemic concentration of agent (warfarin for example is often titrated in this manner), neutrophil count, white blood cell count, bilirubin concentration or other blood analysis parameters which reflect patient status. The specific characteristics used to determine patient-adjusted dosages are reported for each specific drug within the prescribing information published by the drug sponsor. Additional characteristics for patient-adjusted dosing may be found in the general literature pertaining to specific drugs.

Dosage volume refers to the total volume of a solution of one or more therapeutic agents for intravenous infusion that is dispensed for the patient in order to achieve the patient-adjusted dose. The dosage volume may be presented in one or more suitable containers. For example, a patient who requires a patient-adjusted dose of 234 mg of an agent may be infused with a dosage volume of 156 mL of a solution of the agent at a dosage strength of 1.5 mg/mL.

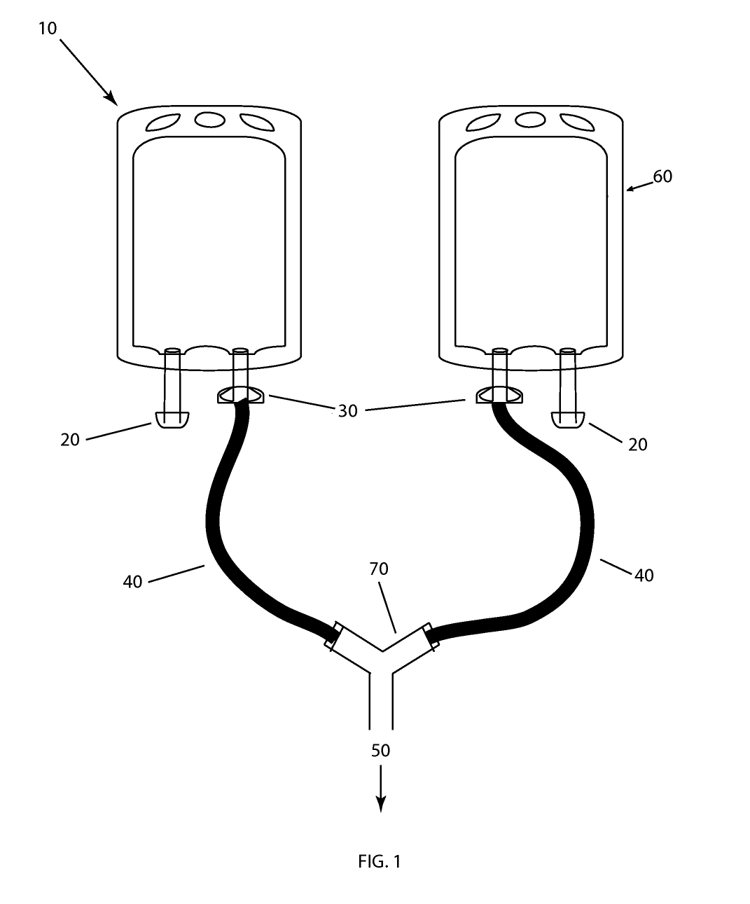

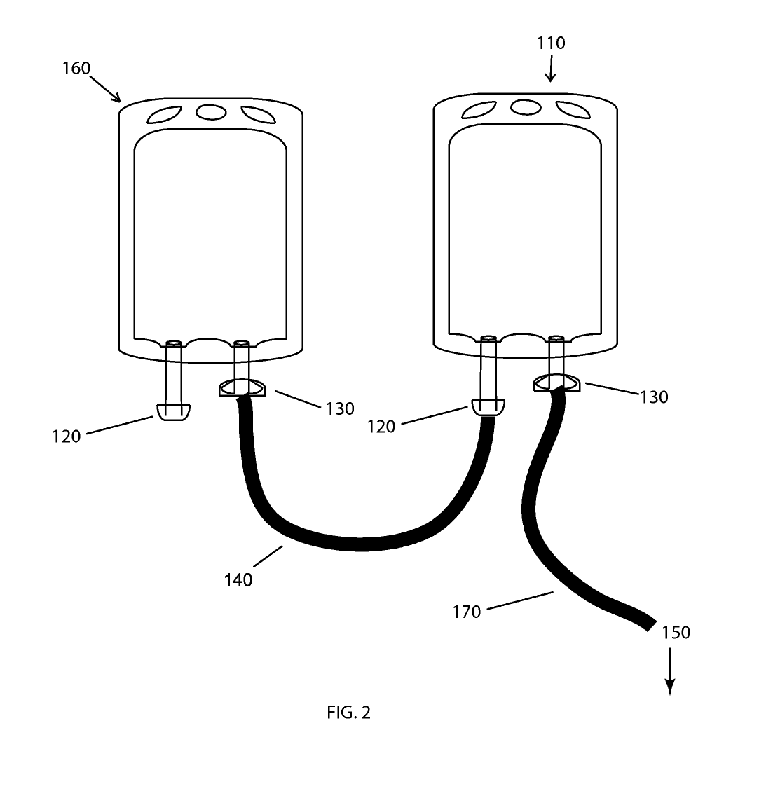

Volume adjustment refers to adding solution to, or withdrawing solution from, one or more patient dosing containers. Top-up volume refers to the volume of solution that should be added to one or more patient dosing containers in order to achieve the dosage volume. In some variations, the top-up volume may be taken from a pharmacy container. Surplus volume refers to the volume of solution that should be withdrawn from one or more patient dosing containers in order to achieve the dosage volume. For example, a particular therapeutic agent may be presented in patient dosing containers of 150 mL and 250 mL. A patient requiring a dosage volume of 450 mL could be treated by combining a 150 mL patient dosing container and a 250 mL patient dosing container and adding a top-up volume of 50 mL from a pharmacy container. The full 50 mL could be added to one of the patient dosing containers, or a portion of the 50 mL could be added to one of the patient dosing containers and the remaining portion added to the other patient dosing containers. Equally, the patient could be treated by combining two patient dosing containers of 250 mL and withdrawing a surplus volume of 50 mL. The full 50 mL could be withdrawn from one of the patient dosing containers, or a portion of the 50 mL could be withdrawn from one or more of the patient dosing containers and the remaining portion could be withdrawn from the other patient dosing container. Alternatively, the patient could be treated by combining three patient dosing containers of 150 mL, which would not require any top-up volume addition or surplus volume withdrawal. Within this context, the term “combining” means to join one or more patient dosing containers together such that the formulation in each container is available to the patient, optionally via some delivery pump. In variations in which there is more than one patient dosing container, the patient dosing containers may be joined in parallel (FIG. 1) or in series (FIG. 2), or given sequentially one after the other to the patient in need. The top-up volume may be added to, or the surplus volume withdrawn from, one the patient dosing containers, or alternatively, partially added to, or withdrawn from, more than one of the dosing containers. The term “sequentially” means one following the other, generally allowing only for the reasonable time required by administering staff to identify the need for changeover and change dosing containers. The pharmacy containers may be configured to allow transfer of multiple portions of the solution. This may allow the pharmacy containers to be used to transfer solution to the patient dosing bags of more than one patient.

FIG. 1 shows an example of a first patient dosing container (10) and a second patient dosing container (60) disposed in a parallel configuration. Each of the first and second patient dosing containers (10) and (60) in this example may be fitted with a first port (20) to facilitate the addition or withdrawal of formulation. The first port (20) may be a conventional septum device designed to be pierced by a syringe needle, or a needle free valve suitable for the purpose. The first and second patient dosing containers (10) and (60) may each be further equipped with a second port (30) designed for connecting with a “giving set” (40). Each giving set (40) may be connected to a piggy-back device (70), which may optionally contain a one way valve device. The piggy-back device (70) may be further connected to the patient (50), either directly by a further giving set, or by means of an infusion pump device.

FIG. 2 shows an example of a first patient dosing container (110) and a second patient dosing container (160) connected in series such that the formulation from the second patient dosing container (160) flows into first patient dosing container (110) via connecting tube (140). Each of the first and second dosing containers (110) and (160) in this example may be fitted with a first port (120) to facilitate the addition or withdrawal of formulation. The first port (120) may be a conventional septum device designed to be pierced by a syringe needle, or a needle free valve suitable for the purpose. The first and second patient dosing containers (110) and (160) may each be further equipped with a second port (130) designed for allowing the efflux of formulation. A patient connecting tube (170) attached to patient dosing container (110) via second port (130) may be further connected to an infusion pump device or directly to the patient (150).

The term “hepatic function” is used to describe the relative ability of the liver to perform its normal functions. A patient's hepatic function can be affected by liver disease or hepatic disease, which is used to describe any disorder of the liver that affects its ability to function properly. There are over 100 different types of liver disease including hepatitis, cirrhosis, fatty liver disease and cancer. Liver disease can be hereditary, the result of drug/alcohol abuse or can even be caused by viruses in the case of hepatitis A, B and C. The most common tests for hepatic function include: AST (Aspartate transaminase) or ALT (alanine aminotransferase), Bilirubin, GGT (Gamma glutamyl transpeptidase), LDH (Lactate dehydrogenase) and Bile Acids.

The term “renal function” is used to describe the relative ability of the kidneys to perform their normal function. A patient's renal function can be affected by kidney disease or renal disease, which is used to describe any disorder of the kidney that affects its ability to function properly. Well-known tests for kidney disease include measuring serum creatinine, glomerular filtration rate, microalbumin, blood urea nitrogen, hemoglobin, hematocrit, and the like.

In one embodiment of the invention, according to Example 1 described herein, the manufacturer prepares in bulk 1) a dosing bag containing 150 mL of 5-fluorouracil in 5% glucose at a concentration of 3.5 mg/mL; 2) a dosing bag containing 250 mL of 5-fluorouracil in 5% glucose at a concentration of 3.5 mg/mL; and 3) a pharmacy reservoir bag containing a supply of 5-fluorouracil in 5% glucose at a concentration of 3.5 mg/mL.

### EXEMPLARY EMBODIMENTS

### Embodiment 1

In one embodiment, the invention provides a method for intravenous delivery of a patient-adjusted dose of a therapeutic agent in a solution of predetermined concentration to a patient in need thereof, comprising: administering intravenously to the patient in need thereof a dosage volume of the solution having a predetermined concentration of the therapeutic agent, wherein the predetermined concentration of the therapeutic agent is used for each patient treated with the therapeutic agent at a particular dosage strength, and wherein the predetermined concentration of the therapeutic agent is independent of the patient-adjusted dose, and wherein the dosage volume is selected to deliver the patient-adjusted dose to the patient.

### Embodiment 2

In a further embodiment of embodiment 1, the patient-adjusted dose of the therapeutic agent is a function of at least one patient characteristic.

### Embodiment 3

In a further embodiment of embodiment 2, the patient characteristic is selected from the group consisting of the patient's body surface area, the patient's body weight or ideal body weight, the patient's hepatic function, and the patient's renal function.

### Embodiment 4

In a further embodiment of embodiment 2, the patient characteristic is the patient's body surface area.

### Embodiment 5

In a further embodiment of embodiment 2, the patient characteristic is patient's body weight or ideal body weight.

### Embodiment 6

In a further embodiment of embodiment 2, the patient characteristic is the patient's hepatic function.

### Embodiment 7

In a further embodiment of embodiment 2, the patient characteristic is the patient's renal function.

### Embodiment 8

In a further embodiment of any one of embodiments 1-7, the therapeutic agent is selected from the group consisting of an antibiotic, an antiviral, a muscle relaxant, a sedative, an anesthetic, a cancer chemotherapeutic agent, and an antifungal. In a further embodiment of embodiment 8, the therapeutic agent is a cancer chemotherapeutic agent.

### Embodiment 9

In a further embodiment of any one of embodiments 1-7, the therapeutic agent is selected from the group consisting of irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine.

### Embodiment 10

In a further embodiment of any one of embodiments 1-7, the therapeutic agent is a glucocorticoid.

### Embodiment 11

In a further embodiment of any one of embodiments 1-7 or 10, the therapeutic agent is selected from the group consisting of dexamethasone and cortisol.

### Embodiment 12

In a further embodiment of any one of embodiments 1-7, the therapeutic agent comprises semi-synthetic irinotecan.

### Embodiment 13

In a further embodiment of any one of embodiments 1-7, the therapeutic agent comprises irinotecan.

### Embodiment 14

In a further embodiment of any one of embodiments 1-13, the predetermined concentration of the therapeutic agent in the solution is a therapeutically effective concentration.

### Embodiment 15

In a further embodiment of any one of embodiments 1-9 or 14-15, the therapeutic agent comprises irinotecan, and wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL.

### Embodiment 16

In a further embodiment of any one of embodiments 1-9 or 14-15, the therapeutic agent comprises irinotecan, and wherein the predetermined concentration is 0.72 mg/mL.

### Embodiment 17

In a further embodiment of any one of embodiments 1-9 or 14-15, the therapeutic agent comprises irinotecan, and wherein the predetermined concentration is 0.6-0.8 mg/mL.

### Embodiment 18

In a further embodiment of any one of embodiments 1-9 or 14-15, the therapeutic agent comprises irinotecan, and wherein the predetermined concentration is 0.6 mg/mL.

### Embodiment 19

In a further embodiment of any one of embodiments 1-9 or 14-15, the therapeutic agent comprises irinotecan, and wherein the predetermined concentration is 0.5-0.7 mg/mL.

### Embodiment 20

In a further embodiment of any one of embodiments 1-9 or 14-15, the therapeutic agent comprises irinotecan, and wherein the predetermined concentration is 0.48 mg/mL.

### Embodiment 21

In a further embodiment of any one of embodiments 1-9 or 14-15, the therapeutic agent comprises irinotecan, and wherein the predetermined concentration is 0.4-0.6 mg/mL.

### Embodiment 22

In a further embodiment of any one of embodiments 1-21, the solution of predetermined concentration of the therapeutic agent further comprises one or more excipients or adjuvants.

### Embodiment 23

In a further embodiment of embodiment 22, the one or more excipients or adjuvants are selected from the group consisting of sodium hyaluronate, lactic acid, sorbitol, and glucose. In a further embodiment of embodiment 22, the adjuvant is present and is sodium hyaluronate.

### Embodiment 24

In a further embodiment of any one of embodiments 1-23, the solution of predetermined concentration of the therapeutic agent further comprises a diluent, wherein the diluent is selected from the group consisting of saline, Ringer's solution, and glucose solution.

### Embodiment 25

In a further embodiment of embodiment 24, the diluent comprises five percent glucose solution.

### Embodiment 26

In a further embodiment of any one of embodiments 1-25, the patient is a mammal.

### Embodiment 27

In a further embodiment of embodiment 26, the mammal is selected from the group consisting of bovine, canine, equine, feline, porcine, and human. In a further embodiment of embodiment 26 the mammal is human.

### Embodiment 28

In one embodiment, the invention provides a system for intravenous delivery of a patient-adjusted dose of a therapeutic agent in a solution of predetermined concentration to a patient in need thereof of any one of the embodiments of embodiments 1-27, comprising at least one pharmacy container containing the solution of predetermined concentration of the therapeutic agent, wherein the at least one pharmacy container is configured to allow transfer of multiple portions of the solution; one or more patient dosing containers each containing an initial volume of the solution of predetermined concentration of the therapeutic agent, wherein the one or more patient dosing containers are configured to allow volume adjustment and are configured for intravenous administration of the solution to the patient, wherein each of the one or more patient dosing containers may contain the same or different initial volumes of the solution, and wherein the predetermined concentration of the solution in the at least one pharmacy container and the one or more patient dosing containers is the same concentration.

### Embodiment 29

In a further embodiment of embodiment 28, the volume adjustment comprises introduction of a top-up volume of the solution from the at least one pharmacy container into one or more of the one or more patient dosing containers.

### Embodiment 30

In a further embodiment of embodiment 28, the volume adjustment comprises withdrawal of a surplus volume of the solution from one or more of the patient dosing containers.

### Embodiment 31

In a further embodiment of embodiment 28, the volume adjustment comprises introduction of a top-up volume of the solution from the at least one pharmacy container into at least one patient dosing container, and withdrawal of a surplus volume of the solution from at least one other patient dosing container.

### Embodiment 32

In a further embodiment of any one of embodiments 28-31, further comprising instructions for preparing the patient-adjusted dose of the therapeutic agent, wherein the instructions indicate that the patient-adjusted dose of the therapeutic agent can be prepared using a specified volume of the solution of predetermined concentration.

### Embodiment 33

In a further embodiment of embodiment 32, the instructions further describe the specified volume of the solution of predetermined concentration required to provide the patient-adjusted dose of the therapeutic agent as a function of at least one patient characteristic.

### Embodiment 34

In a further embodiment of embodiment 33, the patient characteristic is selected from the group consisting of the patient's body surface area, the patient's body weight or ideal body weight, the patient's hepatic function, and the patient's renal function.

### Embodiment 35

In a further embodiment of embodiment 33, the patient characteristic is the patient's body surface area.

### Embodiment 36

In a further embodiment of embodiment 33, the patient characteristic is patient's body weight or ideal body weight.

### Embodiment 37

In a further embodiment of embodiment 33, the patient characteristic is the patient's hepatic function.

### Embodiment 38

In a further embodiment of embodiment 33, the patient characteristic is the patient's renal function.

### Embodiment 39

In a further embodiment of any one of embodiments 28-28, the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume.

### Embodiment 40

In a further embodiment of embodiment 39, further comprising instructions for preparing the patient adjusted dose, wherein the instructions describe the number of patient dosing containers from the first group and the number of patient dosing containers from the second group and the volume, if any, of the solution of predetermined concentration from the pharmacy container required to provide the patient-adjusted dose.

### Embodiment 41

In a further embodiment of any one of embodiments 28-40, the one or more patient dosing containers comprise one or more materials, wherein the one or more materials are selected from the group consisting of plastic and glass.

### Embodiment 42

In a further embodiment of any one of embodiments 28-41, the at least one pharmacy container comprises one or more materials, wherein the one or more materials are selected from the group consistent of plastic and glass.

### Embodiment 43

In a further embodiment of embodiment 39 or 40, the one or more materials comprise polypropylene.

### Embodiment 44

In a further embodiment of any one of embodiments 28-43, the one or more patient dosing containers comprise triple layer polypropylene bags.

### Embodiment 45

In a further embodiment of any one of embodiments 28-44, the at least one pharmacy container comprises a triple layer polypropylene bag.

### Embodiment 46

In a further embodiment of any one of embodiments 26-42, the at least one pharmacy container comprises a syringe.

### Embodiment 47

In a further embodiment of any one of embodiments 28-46, the one or more patient dosing containers limit light transmission to the solution.

### Embodiment 48

In a further embodiment of any one of embodiments 28-47, the one or more patient dosing containers comprise on or more materials that reflect light.

### Embodiment 49

In a further embodiment of any one of embodiments 28-48, the one or more patient dosing containers comprise a foil layer.

### Embodiment 50

In a further embodiment of any one of embodiments 28-49, the one or more patient dosing containers comprise a double-laminated foil package.

### Embodiment 51

In a further embodiment of any one of embodiments 28-50, the one or more patient dosing containers comprise one or more materials that absorb light.

### Embodiment 52

In a further embodiment of any one of embodiments 28-51, the at least one pharmacy container limits light transmission to the solution.

### Embodiment 53

In a further embodiment of any one of embodiments 28-52, the at least one pharmacy container limits light transmission to the solution.

### Embodiment 54

In a further embodiment of any one of embodiments 28-53, the at least one pharmacy container reflects light.

### Embodiment 55

In a further embodiment of any one of embodiments 28-54, the at least one pharmacy container comprises a foil layer.

### Embodiment 56

In a further embodiment of any one of embodiments 28-54, the at least one pharmacy container comprises a double-laminated foil package.

### Embodiment 57

In a further embodiment of any one of embodiments 28-56, the at least one pharmacy container comprises one or more materials that absorb light.

### Embodiment 58

In a further embodiment of any one of embodiments 26-57, the system comprises at least two patient dosing containers, further comprising connecting tubes, wherein the connecting tubes connect the patient dosing containers.

### Embodiment 59

In a further embodiment of embodiment 58, the connecting tubes comprise a small bore Y-extension set.

### Embodiment 60

In a further embodiment of any one of embodiments 28-59, the therapeutic agent is photolabile.

### Embodiment 61

In a further embodiment of any one of embodiments 28-60, the therapeutic agent is selected from the group consisting of an antibiotic, an antiviral, a muscle relaxant, a sedative, an anesthetic, a cancer chemotherapeutic agent, and an antifungal. In a further embodiment of embodiment 61, the therapeutic agent is a cancer chemotherapeutic agent.

### Embodiment 62

In a further embodiment of any one of embodiments 28-60, the therapeutic agent is selected from the group consisting of irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine.

### Embodiment 63

In a further embodiment of any one of embodiments 28-60, the therapeutic agent is a glucocorticoid.

### Embodiment 64

In a further embodiment of any one of embodiments 28-60 and 63, the therapeutic agent is selected from the group consisting of dexamethasone and cortisol.

### Embodiment 65

In a further embodiment of any one of embodiments 28-58, the therapeutic agent comprises semi-synthetic irinotecan.

### Embodiment 66

In a further embodiment of any one of embodiments 28-58, the therapeutic agent comprises irinotecan.

### Embodiment 67

In a further embodiment of any one of embodiments 26-63, the predetermined concentration of the therapeutic agent in the solution is a therapeutically effective concentration.

### Embodiment 68

In a further embodiment of any one of embodiments 28-62 and 66-67, the therapeutic agent comprises irinotecan, and wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL.

### Embodiment 69

In a further embodiment of any one of embodiments 28-62 and 66-68, the therapeutic agent comprises irinotecan, and wherein the predetermined concentration is 0.72 mg/mL.

### Embodiment 70

In a further embodiment of any one of embodiments 28-62 and 66-68, the therapeutic agent comprises irinotecan, and wherein the predetermined concentration is 0.6-0.8 mg/mL.

### Embodiment 71

In a further embodiment of any one of embodiments 28-62 and 66-68, the therapeutic agent comprises irinotecan, and wherein the predetermined concentration is 0.6 mg/mL.

### Embodiment 72

In a further embodiment of any one of embodiments 28-62 and 66-68, the therapeutic agent comprises irinotecan, and wherein the predetermined concentration is 0.5-0.7 mg/mL.

### Embodiment 73

In a further embodiment of any one of embodiments 28-62 and 66-68, the therapeutic agent comprises irinotecan, and wherein the predetermined concentration is 0.48 mg/mL.

### Embodiment 74

In a further embodiment of any one of embodiments 28-62 and 66-68, the therapeutic agent comprises irinotecan, and wherein the predetermined concentration is 0.4-0.6 mg/mL.

### Embodiment 75

In a further embodiment of any one of embodiments 28-74, the solution of predetermined concentration of the therapeutic agent further comprises one or more excipients or adjuvants.

### Embodiment 76

In a further embodiment of embodiment 75, the one or more excipients or adjuvants are selected from the group consisting of sodium hyaluronate, lactic acid, sorbitol, and glucose. In a further embodiment of embodiment 76, the adjuvant is present and is sodium hyaluronate.

### Embodiment 77

In a further embodiment of any one of embodiments 1-76, the solution of predetermined concentration of the therapeutic agent further comprises a diluent, wherein the diluent is selected from the group consisting of saline, Ringer's solution, and glucose solution.

### Embodiment 78

In a further embodiment of embodiment 77, the diluent comprises five percent glucose solution.

### Embodiment 79

In a further embodiment of any one of embodiments 28-78, the patient is a mammal.

### Embodiment 80

In a further embodiment of embodiment 79, the mammal is selected from the group consisting of bovine, canine, equine, feline, porcine, and human. In a further embodiment of embodiment 80, the mammal is human.

### Embodiment 81

In one embodiment, the invention provides a method of preparing a patient-adjusted dose of a therapeutic agent for intravenous delivery to a patient in need thereof using the embodiment of any one of embodiments 28-80, comprising determining a dosage volume of the solution of predetermined concentration of the therapeutic agent to be administered to the patient, wherein the dosage volume is the volume of solution required to provide the patient-adjusted dose; and selecting one or more patient dosing containers containing the solution of predetermined concentration of the therapeutic agent, wherein if the total volume in the one or more patient dosing containers is less than the dosage volume, then adding a top-up volume of the solution of predetermined concentration of the therapeutic agent from a pharmacy container to one or more of the one or more patient dosing containers, such that after addition of the top-up volume, the total volume of solution in the one or more patient dosing containers is equal to the dosage volume, wherein the predetermined concentration of the solution in the pharmacy container and the one or more patient dosing containers is the same concentration, and wherein the pharmacy container is configured to allow transfer of multiple portions of the solution, or if the total volume in the one or more patient dosing containers is more than the dosage volume, then withdrawing a surplus volume of the solution of predetermined concentration from one or more of the one or more patient dosing containers, such that after withdrawal of the surplus volume, the total volume of solution in the one or more patient dosing containers is equal to the dosage volume, wherein the one or more patient dosing containers are configured to allow volume adjustment and are configured for intravenous administration of the solution to the patient.

### Embodiment 82

In a further embodiment of embodiment 81, the total volume in the one or more patient dosing containers is less than the dosage volume.

### Embodiment 83

In a further embodiment of embodiment 82, volume adjustment comprises introduction of a top-up volume of the solution from at least one pharmacy container into one or more of the one or more patient dosing containers.

### Embodiment 84

In a further embodiment of embodiment 81, the total volume in the one or more patient dosing containers is more than the dosage volume.

### Embodiment 85

In a further embodiment of embodiment 84, volume adjustment comprises withdrawal of a surplus volume of the solution from one or more of the patient dosing containers.

### Embodiment 86

In a further embodiment of any one of embodiments 81-82 and 84, the volume adjustment comprises introduction of a top-up volume of the solution from the at least one pharmacy container into at least one patient dosing container, and withdrawal of a surplus volume of the solution from at least one other patient dosing container.

### Embodiment 87

In a further embodiment of any one of embodiments 81-86, the patient-adjusted dose of the therapeutic agent is a function of at least one patient characteristic.

### Embodiment 88

In a further embodiment of embodiment 87, the patient characteristic is selected from the group consisting of the patient's body surface area, the patient's body weight or ideal body weight, the patient's hepatic function, and the patient's renal function.

### Embodiment 89

In a further embodiment of embodiment 87, the patient characteristic is the patient's body surface area.

### Embodiment 90

In a further embodiment of embodiment 87, the patient characteristic is patient's body weight or ideal body weight.

### Embodiment 91

In a further embodiment of embodiment 87, the patient characteristic is the patient's hepatic function.

### Embodiment 92

In a further embodiment of embodiment 87, the patient characteristic is the patient's renal function.

### Embodiment 93

In a further embodiment of any one of embodiments 81-92, the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume.

### Embodiment 94

In a further embodiment of any one of embodiments 81-93, the one or more patient dosing containers comprise one or more materials, wherein the one or more materials are selected from the group consisting of plastic and glass.

### Embodiment 95

In a further embodiment of any one of embodiments 81-94, the at least one pharmacy container comprises one or more materials, wherein the one or more materials are selected from the group consistent of plastic and glass.

### Embodiment 96

In a further embodiment of embodiment 94 or 95, the one or more materials comprise polypropylene.

### Embodiment 97

In a further embodiment of any one of embodiments 81-96, the one or more patient dosing containers comprise triple layer polypropylene bags.

### Embodiment 98

In a further embodiment of any one of embodiments 81-97, the at least one pharmacy container comprises a triple layer polypropylene bag.

### Embodiment 99

In a further embodiment of any one of embodiments 81-97, the at least one pharmacy container comprises a syringe.

### Embodiment 100

In a further embodiment of any one of embodiments 81-99, the one or more patient dosing containers limit light transmission to the solution.

### Embodiment 101

In a further embodiment of any one of embodiments 81-100, the one or more patient dosing containers comprise on or more materials that reflect light.

### Embodiment 102

In a further embodiment of any one of embodiments 81-101, the one or more patient dosing containers comprise a foil layer.

### Embodiment 103

In a further embodiment of any one of embodiments 81-102, the one or more patient dosing containers comprise a double-laminated foil package.

### Embodiment 104

In a further embodiment of any one of embodiments 81-103, the one or more patient dosing containers comprise one or more materials that absorb light.

### Embodiment 105

In a further embodiment of any one of embodiments 81-104, the at least one pharmacy container limits light transmission to the solution.

### Embodiment 106

In a further embodiment of any one of embodiments 81-105, the at least one pharmacy container limits light transmission to the solution.

### Embodiment 107

In a further embodiment of any one of embodiments 81-106, the at least one pharmacy container reflects light.

### Embodiment 108

In a further embodiment of any one of embodiments 81-107, the at least one pharmacy container comprises a foil layer.

### Embodiment 109

In a further embodiment of any one of embodiments 81-108, the at least one pharmacy container comprises a double-laminated foil package.

### Embodiment 110

In a further embodiment of any one of embodiments 81-109, the at least one pharmacy container comprises one or more materials that absorb light.

### Embodiment 111

In a further embodiment of any one of embodiments 81-110, the therapeutic agent is photolabile.

### Embodiment 112

In a further embodiment of any one of embodiments 81-111, the therapeutic agent is selected from the group consisting of an antibiotic, an antiviral, a muscle relaxant, a sedative, an anesthetic, a cancer chemotherapeutic agent, and an antifungal. In a further embodiment of embodiment 112, the therapeutic agent is a cancer chemotherapeutic agent.

### Embodiment 113

In a further embodiment of any one of embodiments 81-111, the therapeutic agent is selected from the group consisting of irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine.

### Embodiment 114

In a further embodiment of any one of embodiments 81-111, the therapeutic agent is a glucocorticoid.

### Embodiment 115

In a further embodiment of any one of embodiments 81-111 and 114, the therapeutic agent is selected from the group consisting of dexamethasone and cortisol.

### Embodiment 116

In a further embodiment of any one of embodiments 81-113, the therapeutic agent comprises semi-synthetic irinotecan.

### Embodiment 117

In a further embodiment of any one of embodiments 81-113, the therapeutic agent comprises irinotecan.

### Embodiment 118

In a further embodiment of any one of embodiments 81-117, the predetermined concentration of the therapeutic agent in the solution is a therapeutically effective concentration.

### Embodiment 119

In a further embodiment of any one of embodiments 81-113 and 117-118, the therapeutic agent comprises irinotecan, and wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL.

### Embodiment 120

In a further embodiment of any one of embodiments 81-113 and 117-118, the therapeutic agent comprises irinotecan, and wherein the predetermined concentration is 0.72 mg/mL.

### Embodiment 121

In a further embodiment of any one of embodiments 81-113 and 117-118, the therapeutic agent comprises irinotecan, and wherein the predetermined concentration is 0.6-0.8 mg/mL.

### Embodiment 122

In a further embodiment of any one of embodiments 81-113 and 117-118, the therapeutic agent comprises irinotecan, and wherein the predetermined concentration is 0.6 mg/mL.

### Embodiment 123

In a further embodiment of any one of embodiments 81-113 and 117-118, the therapeutic agent comprises irinotecan, and wherein the predetermined concentration is 0.5-0.7 mg/mL.

### Embodiment 124

In a further embodiment of any one of embodiments 81-113 and 117-118, the therapeutic agent comprises irinotecan, and wherein the predetermined concentration is 0.48 mg/mL.

### Embodiment 125

In a further embodiment of any one of embodiments 81-113 and 117-118, the therapeutic agent comprises irinotecan, and wherein the predetermined concentration is 0.4-0.6 mg/mL.

### Embodiment 126

In a further embodiment of any one of embodiments 81-125, the solution of predetermined concentration of the therapeutic agent further comprises one or more excipients or adjuvants.

### Embodiment 127

In a further embodiment of embodiment 126, the one or more excipients or adjuvants are selected from the group consisting of sodium hyaluronate, lactic acid, sorbitol, and glucose. In a further embodiment of embodiment 127, the adjuvant is present and is sodium hyaluronate.

### Embodiment 128

In a further embodiment of any one of embodiments 81-127, the solution of predetermined concentration of the therapeutic agent further comprises a diluent, wherein the diluent is selected from the group consisting of saline, Ringer's solution, and glucose solution.

### Embodiment 129

In a further embodiment of embodiment 128, the diluent comprises five percent glucose solution.

### Embodiment 130

In a further embodiment of any one of embodiments 81-129, the patient is a mammal.

### Embodiment 131

In a further embodiment of embodiment 130, the mammal is selected from the group consisting of bovine, canine, equine, feline, porcine, and human. In a further embodiment of embodiment 131, the mammal is human.

### Embodiment 132

In one embodiment, the invention provides a method of manufacturing the embodiment of any one of embodiment 28-80 for intravenous delivery of a patient-adjusted dose of a therapeutic agent in a solution of predetermined concentration to a patient in need thereof, comprising formulating a solution for intravenous delivery, wherein the solution comprises a predetermined concentration of the therapeutic agent; and packaging the solution in a plurality of containers, wherein the plurality of containers comprise one or more patient dosing containers and at least one pharmacy container, wherein the one or more patient dosing containers are configured to allow volume adjustment and are configured for intravenous administration of the solution to a patient, and wherein the at least one pharmacy container is configured to allow transfer of multiple portions of the solution.

### Embodiment 133

In a further embodiment of embodiment 132, volume adjustment comprises introduction of a top-up volume of the solution from at least one pharmacy container into one or more of the one or more patient dosing containers.

### Embodiment 134

In a further embodiment of embodiment 132, the volume adjustment comprises withdrawal of a surplus volume of the solution from one or more of the patient dosing containers.

### Embodiment 135

In a further embodiment of embodiment 132, where the volume adjustment comprises introduction of a top-up volume of the solution from the at least one pharmacy container into at least one patient dosing container, and withdrawal of a surplus volume of the solution from at least one other patient dosing container.

### Embodiment 136

In a further embodiment of any one of embodiments 132-136, the patient-adjusted dose of the therapeutic agent is a function of at least one patient characteristic.

### Embodiment 137

In a further embodiment of embodiment 136, the patient characteristic is selected from the group consisting of the patient's body surface area, the patient's body weight or ideal body weight, the patient's hepatic function, and the patient's renal function.

### Embodiment 138

In a further embodiment of embodiment 136, the patient characteristic is the patient's body surface area.

### Embodiment 139

In a further embodiment of embodiment 136, the patient characteristic is patient's body weight or ideal body weight.

### Embodiment 140

In a further embodiment of embodiment 136, the patient characteristic is the patient's hepatic function.

### Embodiment 141

In a further embodiment of embodiment 136, the patient characteristic is the patient's renal function.

### Embodiment 142

In a further embodiment of any one of embodiments 132-141, the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume.

### Embodiment 143

In a further embodiment of any one of embodiments 132-142, the one or more patient dosing containers comprise one or more materials, wherein the one or more materials are selected from the group consisting of plastic and glass.

### Embodiment 144

In a further embodiment of any one of embodiments 132-143, the at least one pharmacy container comprises one or more materials, wherein the one or more materials are selected from the group consistent of plastic and glass.

### Embodiment 145

In a further embodiment of embodiment 143 or 144, the one or more materials comprise polypropylene.

### Embodiment 146

In a further embodiment of any one of embodiments 132-145, the one or more patient dosing containers comprise triple layer polypropylene bags.

### Embodiment 147

In a further embodiment of any one of embodiments 132-146, the at least one pharmacy container comprises a triple layer polypropylene bag.

### Embodiment 148

In a further embodiment of any one of embodiments 132-146, the at least one pharmacy container comprises a syringe.

### Embodiment 149

In a further embodiment of any one of embodiments 132-148, the one or more patient dosing containers limit light transmission to the solution.

### Embodiment 150

In a further embodiment of any one of embodiments 132-149, the one or more patient dosing containers comprise on or more materials that reflect light.

### Embodiment 151

In a further embodiment of any one of embodiments 132-150, the one or more patient dosing containers comprise a foil layer.

### Embodiment 152

In a further embodiment of any one of embodiments 132-150, the one or more patient dosing containers comprise a double-laminated foil package.

### Embodiment 153

In a further embodiment of any one of embodiments 132-152, the one or more patient dosing containers comprise one or more materials that absorb light.

### Embodiment 154

In a further embodiment of any one of embodiments 132-153, the at least one pharmacy container limits light transmission to the solution.

### Embodiment 155

In a further embodiment of any one of embodiments 132-154, the at least one pharmacy container limits light transmission to the solution.

### Embodiment 156

In a further embodiment of any one of embodiments 132-155, the at least one pharmacy container reflects light.

### Embodiment 157

In a further embodiment of any one of embodiments 132-156, the at least one pharmacy container comprises a foil layer.

### Embodiment 158

In a further embodiment of any one of embodiments 132-157, the at least one pharmacy container comprises a double-laminated foil package.

### Embodiment 159

The method of any of one of claims 132-158, the at least one pharmacy container comprises one or more materials that absorb light.

### Embodiment 160

In a further embodiment of any one of embodiments 132-159, the therapeutic agent is photolabile.

### Embodiment 161

In a further embodiment of any one of embodiments 132-160, the therapeutic agent is selected from the group consisting of an antibiotic, an antiviral, a muscle relaxant, a sedative, an anesthetic, a cancer chemotherapeutic agent, and an antifungal. In a further embodiment of embodiment 161, the therapeutic agent is a cancer chemotherapeutic agent.

### Embodiment 162

In a further embodiment of any one of embodiments 132-160, the therapeutic agent is selected from the group consisting of irinotecan, doxorubicin, cis-platin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine.

### Embodiment 163

In a further embodiment of any one of embodiments 132-160, the therapeutic agent is a glucocorticoid.

### Embodiment 164

In a further embodiment of any one of embodiments 132-160 and 163, the therapeutic agent is selected from the group consisting of dexamethasone and cortisol.

### Embodiment 165

In a further embodiment of any one of embodiments 132-162, the therapeutic agent comprises semi-synthetic irinotecan.

### Embodiment 166

In a further embodiment of any one of embodiments 132-162, the therapeutic agent comprises irinotecan.

### Embodiment 167

In a further embodiment of any one of embodiments 132-163, the predetermined concentration of the therapeutic agent in the solution is a therapeutically effective concentration.

### Embodiment 168

In a further embodiment of any one of embodiments 132-162 and 166-167, the therapeutic agent comprises irinotecan, and wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL.

### Embodiment 169

In a further embodiment of any one of embodiments 132-162 and 166-167, the therapeutic agent comprises irinotecan, and wherein the predetermined concentration is 0.72 mg/mL.

### Embodiment 170

In a further embodiment of any one of embodiments 132-162 and 166-167, the therapeutic agent comprises irinotecan, and wherein the predetermined concentration is 0.6-0.8 mg/mL.

### Embodiment 171

In a further embodiment of any one of embodiments 132-162 and 166-167, the therapeutic agent comprises irinotecan, and wherein the predetermined concentration is 0.6 mg/mL.

### Embodiment 172

In a further embodiment of any one of embodiments 132-162 and 166-167, the therapeutic agent comprises irinotecan, and wherein the predetermined concentration is 0.5-0.7 mg/mL.

### Embodiment 173

In a further embodiment of any one of embodiments 132-162 and 166-167, the therapeutic agent comprises irinotecan, and wherein the predetermined concentration is 0.48 mg/mL.

### Embodiment 174

In a further embodiment of any one of embodiments 132-162 and 166-167, the therapeutic agent comprises irinotecan, and wherein the predetermined concentration is 0.4-0.6 mg/mL.

### Embodiment 175

In a further embodiment of any one of embodiments 132-174, the solution of predetermined concentration of the therapeutic agent further comprises one or more excipients or adjuvants.

### Embodiment 176

In a further embodiment of embodiment 175, the one or more excipients or adjuvants are selected from the group consisting of sodium hyaluronate, lactic acid, sorbitol, and glucose. In a further embodiment of embodiment 176, the adjuvant is present and is sodium hyaluronate.

### Embodiment 177

In a further embodiment of any one of embodiments 132-176, the solution of predetermined concentration of the therapeutic agent further comprises a diluent, wherein the diluent is selected from the group consisting of saline, Ringer's solution, and glucose solution.

### Embodiment 178

In a further embodiment of embodiment 177, the diluent comprises five percent glucose solution.

### Embodiment 179

In a further embodiment of any one of embodiments 132-178, the patient is a mammal.

### Embodiment 180

In a further embodiment of embodiment 179, the mammal is selected from the group consisting of bovine, canine, equine, feline, porcine, and human. In a further embodiment of embodiment 180, the mammal is human.

### Embodiment 181

In a further embodiment of any one of embodiments 132-180, packaging the solution in the plurality of containers comprises aseptic filtration of the solution.

### Embodiment 182

In one embodiment, the invention provides use of the embodiment of any one of embodiments 28-80 for the embodiment of any one of embodiments 1-27.

### Embodiment 183

In a further embodiment of any one of embodiments 1-7, 28-58, 81-113, or 132-162, the therapeutic agent comprises synthetic irinotecan.

### Embodiment 184

In one embodiment, the invention provides use of a therapeutic agent in the preparation of a medicament for treating a patient in need thereof, wherein the therapeutic agent is for intravenous administration in a solution having a predetermined concentration of the agent. In certain additional embodiments of embodiment 184, the use incorporates the features described in one or more of embodiments 2 through 27.

### Embodiment 185

In one embodiment, the invention provides use of a therapeutic agent in the preparation of a medicament for treating a patient in need thereof, wherein the agent is for intravenous administration in a solution and wherein a dose is selected for the patient by adjusting the volume of the solution to be administered without adjusting the concentration of the agent therein. In certain additional embodiments of embodiment 185, the use incorporates the features described in one or more of embodiments 2 through 27.

### Embodiment 186

In one embodiment, the invention provides use of a therapeutic agent in the preparation of a medicament for treating a patient in need thereof, wherein the therapeutic agent is for intravenous administration in a solution of predetermined concentration, wherein the predetermined concentration of the therapeutic agent is for use in each patient treated with the therapeutic agent at a particular dosage strength, and wherein the dose is patient-adjusted by adjusting the volume of the solution to be administered. In certain additional embodiments of embodiment 186, the use incorporates the features described in one or more of embodiments 2 through 27.

### Embodiment 187

In one embodiment, the invention provides use of a therapeutic agent in the preparation of a medicament for treating a patient in need thereof, wherein the therapeutic agent is for intravenous delivery in a dosage volume of a solution having a predetermined concentration of the therapeutic agent, wherein the predetermined concentration of the therapeutic agent is for use in each patient treated with the therapeutic agent at a particular dosage strength, and wherein the predetermined concentration of the therapeutic agent is independent of the patient-adjusted dose, and wherein the dosage volume is selected to deliver the patient-adjusted dose to the patient. In certain additional embodiments of embodiment 187, the use incorporates the features described in one or more of embodiments 2 through 27.

### Embodiment 188

In one embodiment, the invention provides a therapeutic agent for use in treating a patient in need thereof wherein the therapeutic agent is for intravenous administration in a solution having a predetermined concentration of the agent. In certain additional embodiments of embodiment 188, the use incorporates the features described in one or more of embodiments 2 through 27.

### Embodiment 189

In one embodiment, the invention provides a therapeutic agent for use in treating a patient in need thereof wherein the therapeutic agent is for intravenous administration in a solution and wherein the dose is selected for the patient by adjusting the volume of the solution to be administered without adjusting the concentration of the agent therein. In certain additional embodiments of embodiment 189, the use incorporates the features described in one or more of embodiments 2 through 27.

### Embodiment 190

In one embodiment, the invention provides a therapeutic agent for use in treating a patient in need thereof wherein the therapeutic agent is for intravenous administration in a solution of predetermined concentration, wherein the predetermined concentration of the therapeutic agent is for use in each patient treated with the therapeutic agent at a particular dosage strength, and wherein the dose is patient-adjusted by adjusting the volume of the solution to be administered. In certain additional embodiments of embodiment 190, the use incorporates the features described in one or more of embodiments 2 through 27.

### Embodiment 191

In one embodiment, the invention provides a therapeutic agent for use in treating a patient in need thereof wherein the therapeutic agent is for intravenous delivery in a dosage volume of a solution having a predetermined concentration of the therapeutic agent, wherein the predetermined concentration of the therapeutic agent is for use in each patient treated with the therapeutic agent at a particular dosage strength, and wherein the predetermined concentration of the therapeutic agent is independent of the patient-adjusted dose, and wherein the dosage volume is selected to deliver the patient-adjusted dose to the patient. In certain additional embodiments of embodiment 191, the use incorporates the features described in one or more of embodiments 2 through 27.

### Embodiment 192

In one embodiment, the invention provides use of a patient-adjusted dose of a therapeutic agent in a solution of predetermined concentration for a patient in need thereof for intravenous delivery, comprising use of a dosage volume of the solution having a predetermined concentration of the therapeutic agent, wherein the predetermined concentration of the therapeutic agent is used for each patient treated with the therapeutic agent at a particular dosage strength, and wherein the predetermined concentration of the therapeutic agent is independent of the patient-adjusted dose, and wherein the dosage volume is selected to deliver the patient-adjusted dose to the patient. In certain additional embodiments of embodiment 192, the use incorporates the features described in one or more of embodiments 2 through 27.

### Embodiment 193

In one embodiment, the invention provides methods of manufacturing a system described herein for intravenous delivery of a patient-adjusted dose of a therapeutic agent in a solution of predetermined concentration to a patient in need thereof, comprising: formulating a solution for intravenous delivery, wherein the solution comprises a predetermined concentration of the therapeutic agent; and packaging the solution in a plurality of containers, wherein the plurality of containers comprise one or more patient dosing containers and at least one pharmacy container, wherein the one or more patient dosing containers are configured to allow volume adjustment and are configured for intravenous administration of the solution to a patient, and wherein the at least one pharmacy container is configured to allow transfer of multiple portions of the solution. In certain additional embodiments of embodiment 193, the system incorporates the systems described in one or more of embodiments 28-80.

194. In one embodiment, the invention provides use of the systems described herein for the intravenous delivery of a patient-adjusted dose of a therapeutic agent in a solution of predetermined concentration to a patient in need thereof. In certain additional embodiments of embodiment 194, the system incorporates the systems described in one or more of embodiments 28-80.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. Preferences and options for a given aspect, feature or parameter of the invention should, unless the context indicates otherwise, be regarded as having been disclosed in combination with any and all preferences and options for all other aspects, features and parameters of the invention. For example, the invention may provide a system and method for dosing with HA-irinotecan. The system and methods may be used for one typical dosing regimen of HA-irinotecan, which consists of a starting dose of 180 mg/m², with dose reductions to 150 mg/m² or 120 mg/m² in response to adverse systemic toxicity. A formulation of irinotecan at a concentration of 0.72 mg/mL and sodium hyaluronate at 4.0 mg/mL in 5% glucose may be used to treat patients on the 180 mg/m² regimen, a formulation of irinotecan at a concentration of 0.60 mg/mL and sodium hyaluronate at 4.0 mg/mL in 5% glucose may be used to treat patients on the 150 mg/m² regimen, and a formulation of irinotecan at a concentration of 0.48 mg/mL and sodium hyaluronate at 4.0 mg/mL in 5% glucose may be used to treat patients on the 120 mg/m² regimen. Each solution may be presented in two different patient dosing containers: a 300 mL polypropylene IV bag filled with 200 mL of formulation and a 250 mL polypropylene IV bag filled with 150 mL of formulation. The solution may also be presented in a pharmacy container: a pharmacy bag containing 200 mL of formulation. The irinotecan dose in mg, and the corresponding volume of formulation needed, may be calculated based on the BSA of the patient and the dose regimen. A combination of one or more patient dosing bags may be selected in order to most closely approach the required volume, and then the required “top-up” volume from the pharmacy bag may be added to one or more of the patient dosing bags, or a surplus volume of formulation may be withdrawn from one or more patient dosing bags using a clean, sterile syringe of suitable volume. In variations in which more than one patient dosing container is selected, the patient dosing bags may be joined in parallel or in series, or given sequentially one after another to the patient in need.

Further details of the invention are illustrated by the following non-limiting Examples. The disclosures of all references in the specification are expressly incorporated herein by reference.

### EXAMPLES

### 1. System and Method for Dosing with 5-fluorouracil

According to the manufacturer's product information leaflet, 5-fluorouracil is typically given at a dose of 15 mg/kg diluted in 300 to 500 mL of 5% glucose solution. Therefore, patients in the weight range of 50 kg to 110 kg will receive a dose concentration of between 1.5 mg/mL and 5.5 mg/mL. In one form of the invention, the manufacturer prepares in bulk 1) a patient dosing bag containing 150 mL of 5-fluorouracil in 5% glucose at a concentration of 3.5 mg/mL; 2) a patient dosing bag containing 250 mL of 5-fluorouracil in 5% glucose at a concentration of 3.5 mg/mL; and 3) a pharmacy bag containing a supply of 5-fluorouracil in 5% glucose at a concentration of 3.5 mg/mL.

A patient of 75 kg body weight would require 1125 mg of 5-fluorouracil in this dosing schedule. This can be delivered by “piggybacking” two 150 mL dosing bag (2*150 mL*3.5 mg/mL=1050 mg) and adding a further 21.5 mL (21.5 mL*3.5 mg/mL=75.25 mg) of top-up volume from the pharmacy bag to one or other of the dosing bags (or partially to one of the patient dosing bags and the remainder to the other patient dosing bag) to give a total dose of 1125.5 mg.

A patient of 95 kg body weight would require 1425 mg of 5-fluorouracil in this dosing schedule. This can be delivered by “piggybacking” a 150 mL patient dosing bag (150 mL*3.5 mg/mL=525 mg) and a 250 mL patient dosing bag (250 mL*3.5 mg/mL=875 mg) and adding a further 7 mL (7 mL*3.5 mg/mL=24.5 mg) of top-up volume from the pharmacy bag to one or other of the patient dosing bags (or partially to one of the patient dosing bags and the remainder to the other patient dosing bag) to give a total dose of 1424.5 mg.

A third patient of 80 kg requires to be dosed at only 10 mg/kg 5-Fluorouracil due to, for example, complications in their treatment. In this case, the patient dose is 800 mg which can be achieved by adding 78.5 mL (274.8 mg) of formulation from a pharmacy bag to a single 150 mL (525 mg) patient dosing bag to provide 799.8 mg of drug. Alternatively, the pharmacist may take a single 250 mL (875 mg) patient dosing bag and withdraw 21.5 mL (75.25 mg) of surplus volume of the formulation, leaving the correct dosage within the bag. The 21.5 mL withdrawn is then suitably disposed of.

### 2. System and Method for Dosing with Irinotecan

According to the manufacturer's product information leaflet for Camptosar®, one typical dosing regimen for irinotecan consists of a starting dose of 180 mg/m² which may be diluted in 5% glucose solution or 0.9% saline solution to a concentration between 0.12 and 2.8 mg/mL. Dose reductions to 150 mg/m² and then 120 mg/m² may be required in response to adverse systemic toxicity.

Table 1 shows the dose of irinotecan in mg required for selected body surface areas at the full dose of 180 mg/m² and the two dose reductions of 150 mg/m² and 120 mg/m². The table also shows the required volume of formulation for these body surface areas at an irinotecan concentration of 1.0 mg/mL.

TABLE 1

| | | | | irinotecan concentration 1.0 mg/mL | | |
|---|---|---|---|---|---|---|
| | | irinotecan | | irinotecan | irinotecan | irinotecan |
| | irinotecan dose | dose (mg) @ | irinotecan dose | volume (mL) | volume (mL) | volume (mL) |
| BSA | (mg) @ 180 mg/m² | 150 mg/m² | (mg) @ 120 mg/m² | @ 180 mg/m² | @ 150 mg/m² | @ 120 mg/m² |
| 2.40 | 432 | 360 | 288 | 432.0 | 360.0 | 288.0 |
| 2.30 | 414 | 345 | 276 | 414.0 | 345.0 | 276.0 |
| 2.20 | 396 | 330 | 264 | 396.0 | 330.0 | 264.0 |
| 2.10 | 378 | 315 | 252 | 378.0 | 315.0 | 252.0 |
| 2.00 | 360 | 300 | 240 | 360.0 | 300.0 | 240.0 |
| 1.90 | 342 | 285 | 228 | 342.0 | 285.0 | 228.0 |
| 1.80 | 324 | 270 | 216 | 324.0 | 270.0 | 216.0 |
| 1.70 | 306 | 255 | 204 | 306.0 | 255.0 | 204.0 |
| 1.60 | 288 | 240 | 192 | 288.0 | 240.0 | 192.0 |
| 1.50 | 270 | 225 | 180 | 270.0 | 225.0 | 180.0 |
| 1.40 | 252 | 210 | 168 | 252.0 | 210.0 | 168.0 |
| 1.30 | 234 | 195 | 156 | 234.0 | 195.0 | 156.0 |

Irinotecan is formulated in 0.9% saline to a concentration of 1.0 mg/mL in the manufacturing facility and the bulk solution is analyzed by quality control according to the relevant pharmacopeia and any relevant specifications for the finished product. The bulk solution is filled to suitable containers, for example a polypropylene IV bag fitted with suitable adapters to be connected to infusion lines and/or infusion pumps. In this example, a 300 mL polypropylene IV bag filled with 200 mL of formulation and a 250 mL polypropylene IV bag filled with 150 mL of formulation are filled. In addition, a 50 mL pre-filled syringe and a 20 mL pre-filled syringe are filled. The pre-filled syringes are suitable sources of “top-up” volume as described below. The IV bags and pre-filled syringes may be filled by aseptic fill techniques with pre-sterilized bulk, or the filled containers may be subject to sterilization after filling.

In use, the pharmacist determines the BSA of the patient and the dose regimen or dose reduction required. This allows the calculation of the dose in mg and the volume of formulation needed. The pharmacist selects a combination of 1 or more IV bags which most closely approaches the required volume and then adds the required “top-up” volume from an appropriate pre-filled syringe. In certain circumstances it might be convenient to select a combination of IV bags which has a volume higher than the required volume. In such cases, a surplus volume of formulation can be withdrawn from a bag using a clean, sterile syringe of suitable volume.

Examples in Practice include:

A patient of 2.40 m² BSA requiring to be dosed at 180 mg/m² (total dose 432 mg, 432 mL) would be suitably treated by taking two 200 mL IV bags and adding 32 mL of “top-up” volume from a 50 mL pre-filled syringe. The two IV bags may be suitably connected by a basic Y-type administration set, such as a small bore Y-extension set, or other appropriate connector.

A patient of 2.40 m² BSA requiring a dose reduction to be dosed at 150 mg/m² (total dose 360 mg, 360 mL) would be suitably treated by taking one 200 mL IV bags and one 150 mL IV bag and adding 10 mL of “top-up” volume from a 20 mL pre-filled syringe. The two IV bags may be suitably connected by a basic Y-type administration set or other appropriate connector.

There are many variations of combinations of bags and top-up volumes which may be suitable. Based on the use of a 200 mL and 150 mL dosing strategy for this product, table 2 outlines potential combinations of patient dosing bags and the required top-up or surplus volumes. Negative volumes indicate removal of a surplus volume of formulation from 1 or more of the IV patient dosing bags, and are highlighted by shading in Tables 2 and 4-8.

TABLE 2

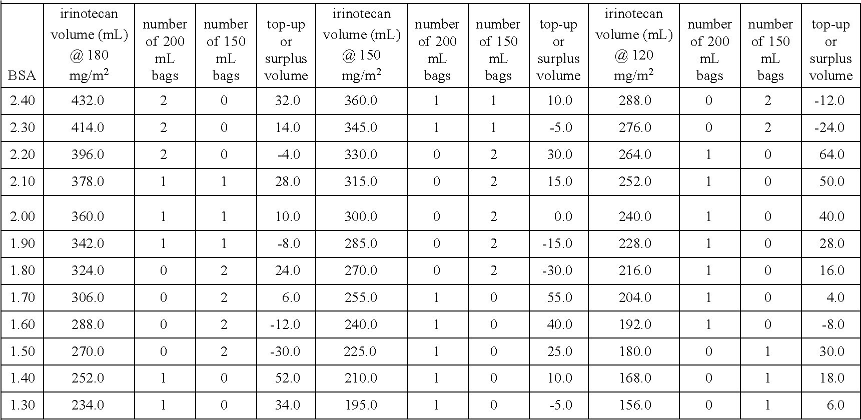

An alternative to the use of pre-filled syringes as a source of the “top-up” volume is to use a patient dosing bag as a reservoir for withdrawing surplus volumes. This arrangement is particularly attractive when multiple doses of therapy are to be prepared at the same time. A further alternative is the provision of a multi-dose pharmacy vial as a source of “top-up” volume.

### 3. System and Method for Dosing with HA-Irinotecan

HA-irinotecan is an innovative formulation comprising an active therapeutic agent—irinotecan, and an active adjuvant—sodium hyaluronate. The irinotecan component is dosed in a manner similar to that shown in example 2—that is, at 180 mg/m² with dose reductions of 150 mg/m² and 120 mg/m². The product dosage differs from irinotecan in example 2 in that while the dose of irinotecan is reduced in response to toxicity, the dose of sodium hyaluronate is not reduced. Consequently, a different formulation of HA-irinotecan is required for each dosage strength since the ratio of sodium hyaluronate to irinotecan changes. In this example, a formulation of irinotecan at a concentration of 0.72 mg/mL and sodium hyaluronate at 4.0 mg/mL in 5% glucose is used to treat patients on the 180 mg/m² regimen, a formulation of irinotecan at a concentration of 0.60 mg/mL and sodium hyaluronate at 4.0 mg/mL in 5% glucose is used to treat patients on the 150 mg/m² regimen and a formulation of irinotecan at a concentration of 0.48 mg/mL and sodium hyaluronate at 4.0 mg/mL in 5% glucose is used to treat patients on the 120 mg/m² regimen. Using these formulations, a patient requiring a dose reduction will receive the same volume but a different formulation strength for each dose reduction—for example: a patient of 2.00 m² will require 360 mg of irinotecan at the dose of 180 mg/m²—this is provided by one 300 mL IV patient dosing bag and one 200 mL IV patient dosing bag giving a total volume of 500 mL at a concentration of 0.72 mg/mL. The same patient, if dose reduced to 150 mg/m² will be treated with 500 mL of formulation at a concentration of 0.60 mg/mL. Similarly, when treated at a dose reduction of 120 mg/m², the same patient will receive 500 mL of 0.48 mg/mL formulation. In this manner the same volume of formulation and configuration of dosing bags can be used for the same BSA. The pharmacist merely selects the correct concentration for the required dosage. Conversely, patients of different BSA within the same dosing regimen (180, 150 or 120 mg/m²) will receive different volumes of the same dosage strength.

Table 3 shows dosing options for a selection of BSA points. In this scenario, every 0.01 m² BSA requires 2.5 mL of formulation. Thus, patients of any body surface area can be effectively treated accurately with this system. In the tabulated example, the large patient dosing bag contains 300 mL of formulation, and the small patient dosing bag contains 200 mL of formulation. A third “pharmacy” bag containing 200 mL of formulation is used as a reservoir for withdrawing the “top-up” volumes required to adjust dosage. The “pharmacy” bag may be sampled multiple times as required, thus reducing the wastage and associated disposal costs for cytotoxic agents.

It can be extrapolated from table 3 that a patient with a BSA of 2.04 m² could be treated by taking one large patient dosing bag (300 mL) and one small patient dosing bag (200 mL) as indicated for a patient of 2.00 m² which is the closest tabulated point and then adding a volume suitable for 0.04 m² (in this case, 10 mL of top-up volume), since every 0.01 m² BSA corresponds to 2.5 mL of formulation.

TABLE 3
Example combinations of one or more pre-filled patient dosing containers
that provide dosage volumes for patients of various body surface areas.

| | number | number | top-up | total | total | total |
|---|---|---|---|---|---|---|
| BSA | large | small | volume | dose | dose | volume |
| m² | bags | bags | ml | mg IRI | gm HA | infused |
| 2.60 | 2 | 0 | 50.0 | 468.0 | 2.60 | 650.0 |
| 2.50 | 2 | 0 | 25.0 | 450.0 | 2.50 | 625.0 |
| 2.40 | 2 | 0 | 0.0 | 432.0 | 2.40 | 600.0 |
| 2.30 | 1 | 1 | 75.0 | 414.0 | 2.30 | 575.0 |
| 2.20 | 1 | 1 | 50.0 | 396.0 | 2.20 | 550.0 |
| 2.10 | 1 | 1 | 25.0 | 378.0 | 2.10 | 525.0 |
| 2.00 | 1 | 1 | 0.0 | 360.0 | 2.00 | 500.0 |
| 1.90 | 0 | 2 | 75.0 | 342.0 | 1.90 | 475.0 |
| 1.80 | 0 | 2 | 50.0 | 324.0 | 1.80 | 450.0 |
| 1.70 | 0 | 2 | 25.0 | 306.0 | 1.70 | 425.0 |
| 1.60 | 0 | 2 | 0.0 | 288.0 | 1.60 | 400.0 |
| 1.50 | 1 | 0 | 75.0 | 270.0 | 1.50 | 375.0 |
| 1.40 | 1 | 0 | 50.0 | 252.0 | 1.40 | 350.0 |
| 1.30 | 1 | 0 | 25.0 | 234.0 | 1.30 | 325.0 |

### 4. System and Method for Dosing with Vancomycin

Vancomycin is a powerful antibiotic which may be given orally or by intravenous infusion to treat certain bacterial infections. When given by intravenous infusion, infusion related events are related to both the concentration and rate of infusion. Concentrations of no more than 5 mg/mL and rates of no more than 10 mg per minute (2 mL per minute) are recommended by Aspen Pharmacare's prescribing information. An initial loading dose of 15 mg/kg of body weight is recommended, followed by maintenance doses of 500 mg every 12 hours or 1 gm every 24 hours for the term of treatment. Renally impaired patients and neonates may be dosed at 10 mg/kg.

The dosing system allows for treatments of initial loading with or without renal impairment, and maintenance dosing.

Bulk formulation of Vancomycin at a concentration of 2.50 mg/mL in 0.9% saline is prepared and filled to IV bags with fill volumes of 100 mL (250 mg); 200 mL (500 mg); and 300 mL (750 mg); as well as a pediatric bag of 60 mL (150 mg). Based on the patient's body weight, the desired number of patient dosing bags of various volumes are selected for infusion according to table 4 below. The appropriate top-up volume is added, or the appropriate surplus volume is withdrawn, to complete the preparation. Maintenance doses consist of one 500 mL bag every 12 hours or two 200 mL bags every 24 hours.

Table 4 shows the required dosage in milligrams, the required dosage volume in mL, the number of various IV bags required and the top-up/surplus volume for the loading dose at 15 mg of Vancomycin per kg of body weight.

Table 5 shows the required dosage in milligrams, the required dosage volume in mL, the number of various IV bags required and the top-up/surplus volume for the loading dose at 10 mg of Vancomycin per kg of body weight as might be required for renally impaired patients and neonates.

TABLE 4
normal loading dose:

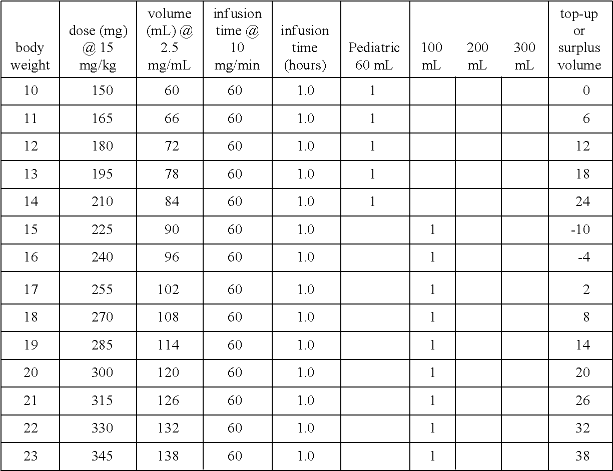

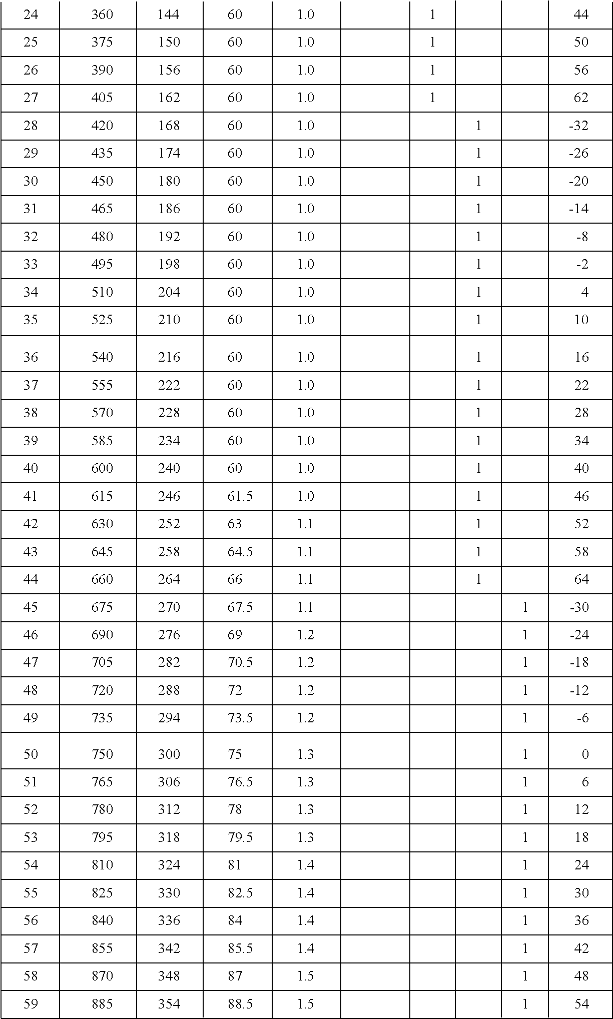

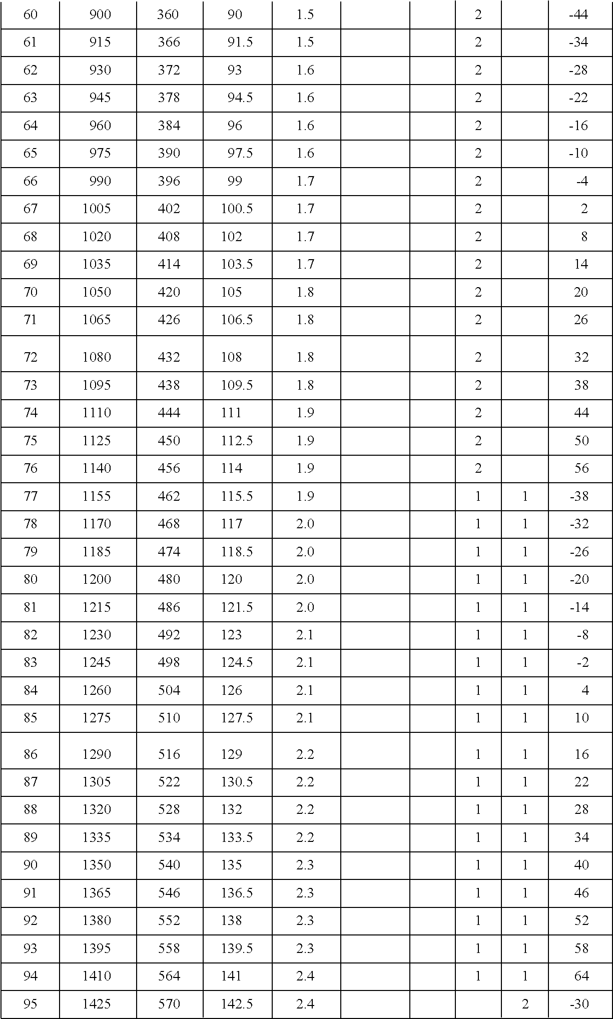

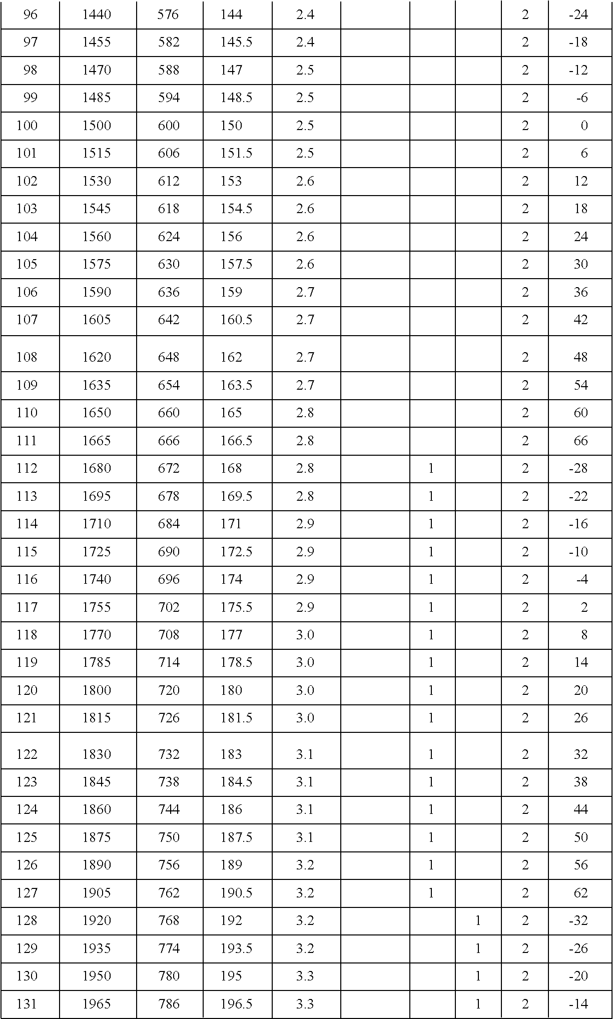

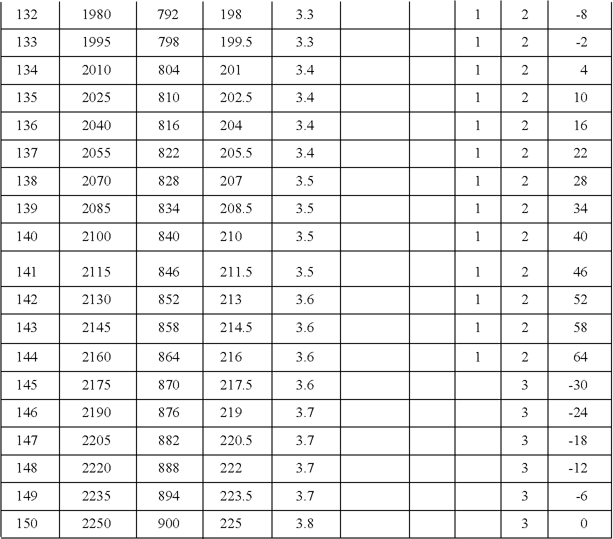

TABLE 5
renally insufficient and neonate loading dose:

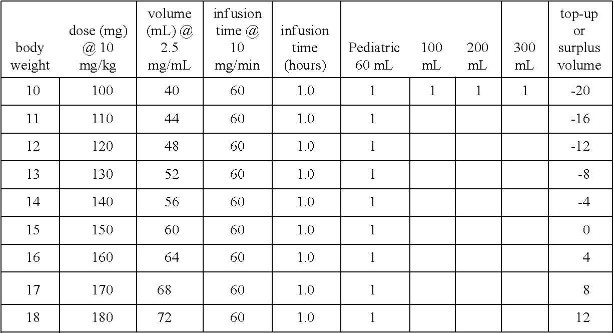

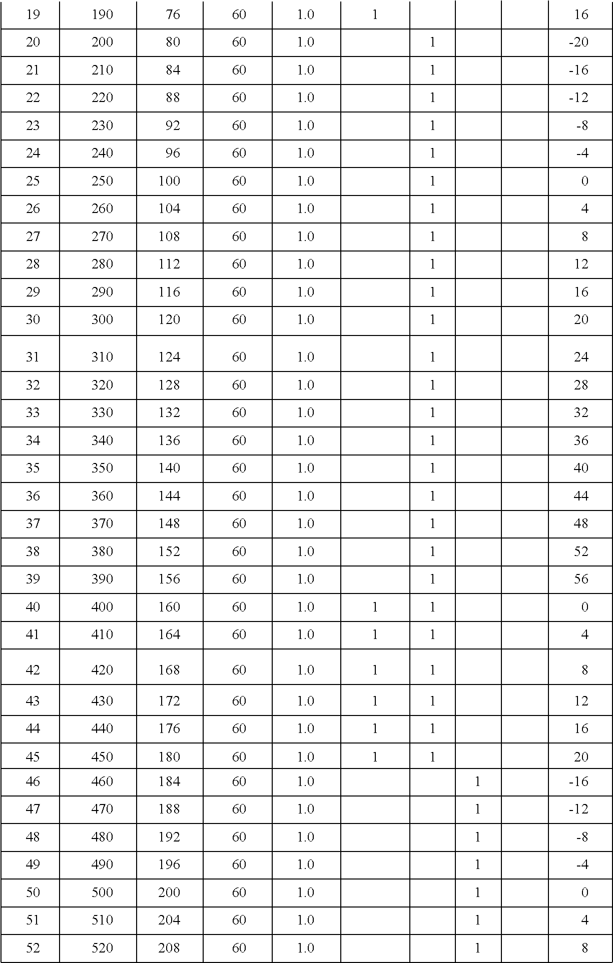

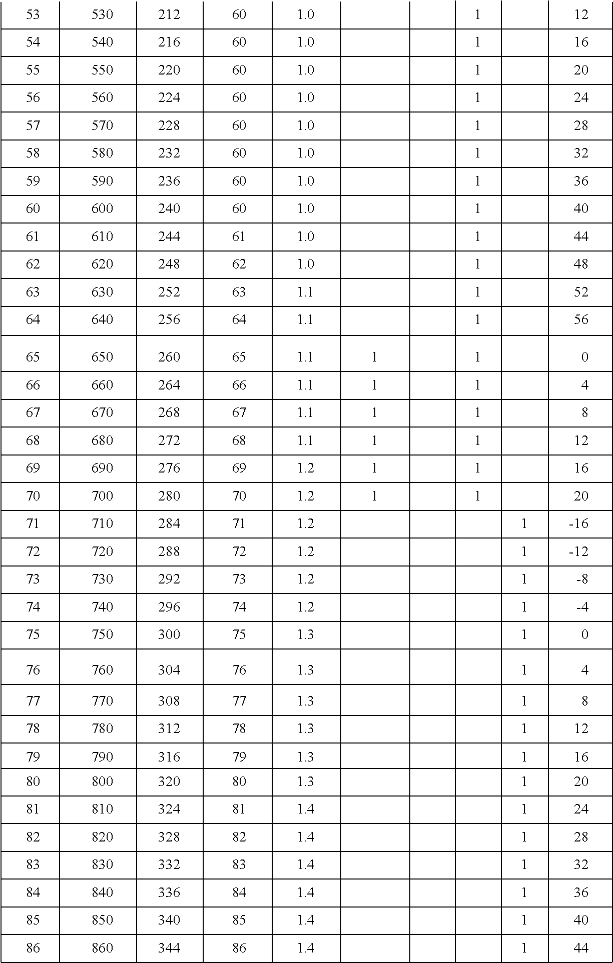

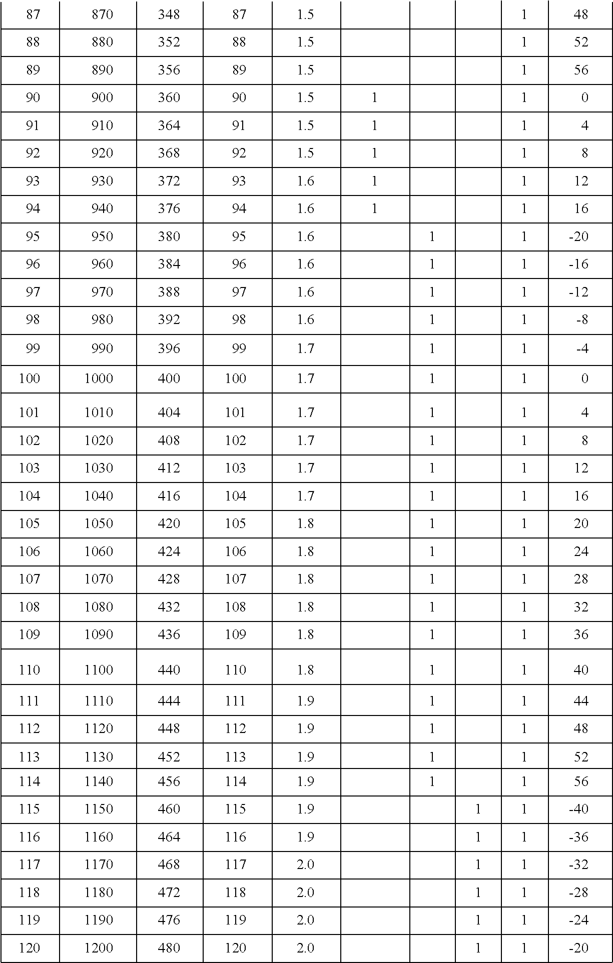

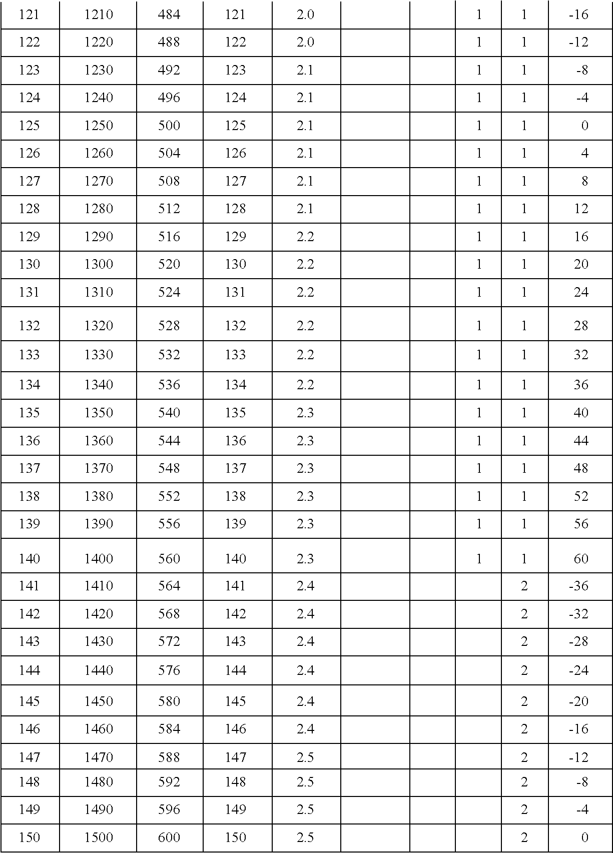

### 5. System and Method for Dosing with Gemcitabine

Gemcitabine is an antineoplastic agent given by intravenous infusion to treat certain cancers. The dose of gemcitabine typically given ranges from 1,250 mg/m² down to 750 mg/m². According to the prescribing information, the drug product is normally dissolved in 0.9% sodium chloride solution, although other solvents may be suitable.

A concentration of 5 mg/mL was selected. At this concentration a small individual (1.30 m²) at a dose of 750 mg/m² would require a volume of 195 mL while a large patient (2.4 m²) at a dose of 1250 mg/m² would require a volume of 600 mL. Thus the system should cater for a range of dose volumes from less than 195 mL through at least 600 mL or greater.

A system comprising three containers was developed. These are a pharmacy container of 100 mL volume containing 500 mg of gemcitabine, a patient dosing container of 200 mL volume (fill volume) containing 1000 mg of gemcitabine and a patient dosing container of 300 mL volume (fill volume) containing 1,500 mg of gemcitabine.

Table 6 shows the required dosage in milligrams, the required dosage volume in mL, the number of various IV bags required and the top-up/surplus volume for the standard dose at 1,000 mg of gemcitabine per m² of BSA. Negative numbers in the top up volume require removal of formulation from one or other (or both) of the patient dosing bags, while positive numbers require addition of top up volume from the pharmacy bag to one or other (or both) of the patient dosing bags.

TABLE 6
standard dosing of gemcitabine

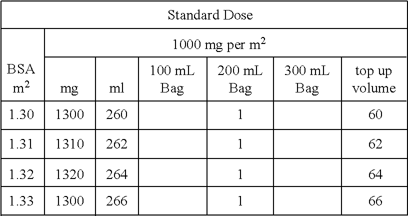

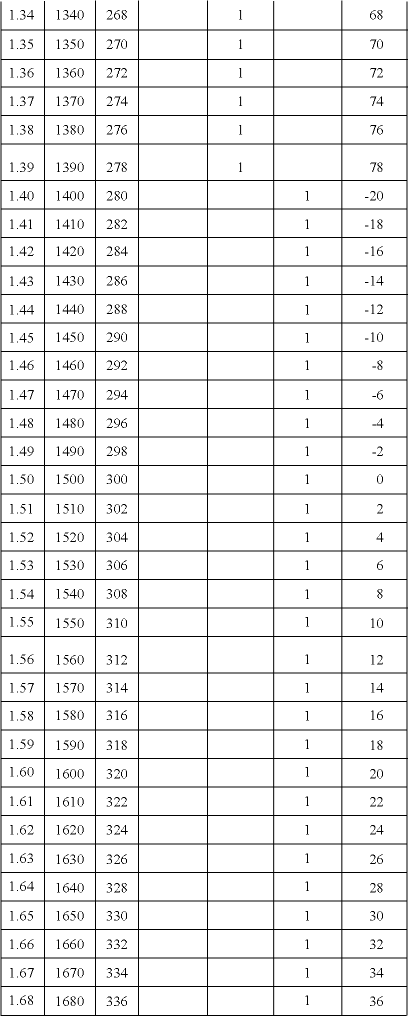

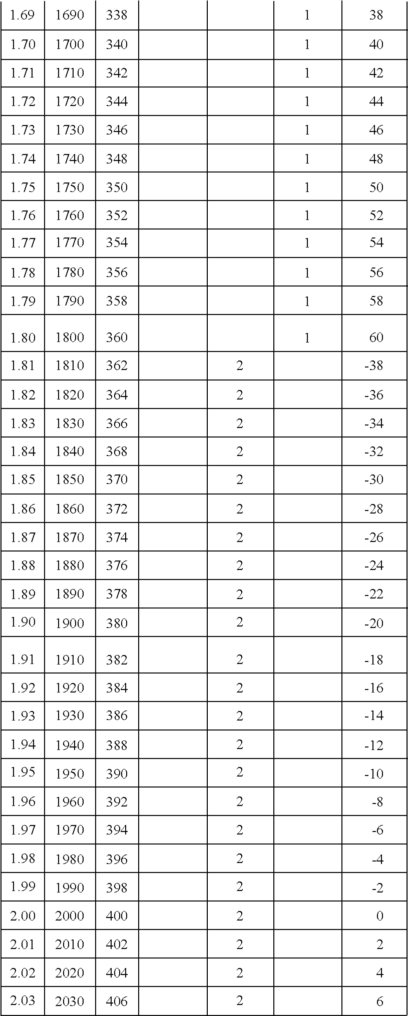

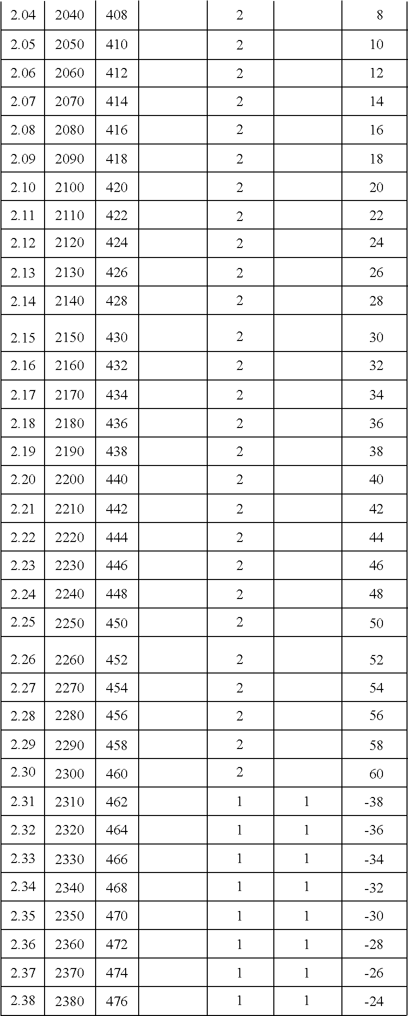

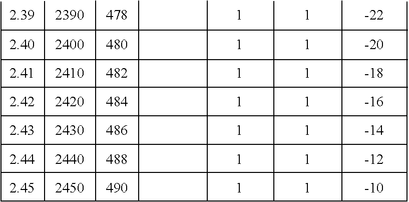

Table 7 shows the required dosage in milligrams, the required dosage volume in mL, the number of various IV bags required and the top-up/surplus volume for a dose of 1250 mg of gemcitabine per m² of BSA.

TABLE 7
increased dose of gemcitabine:

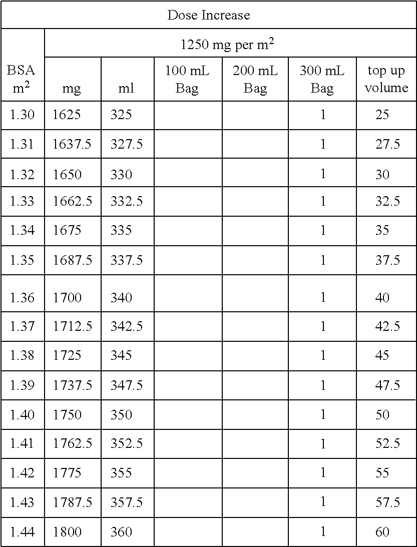

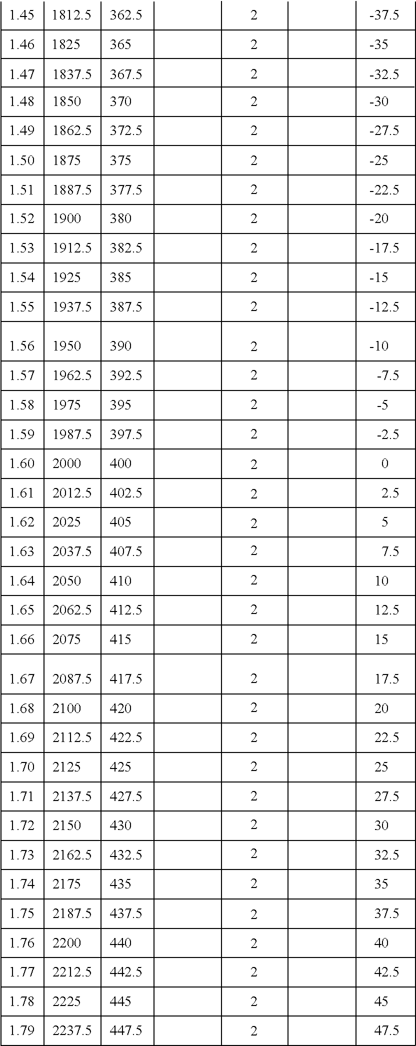

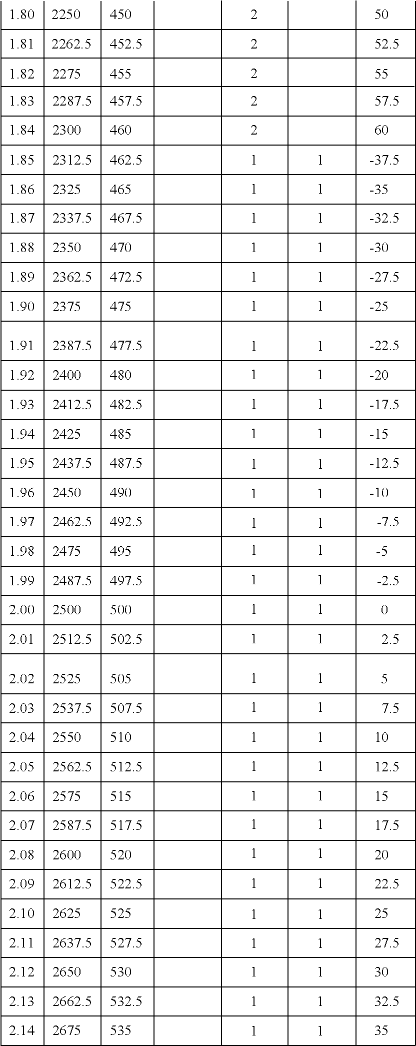

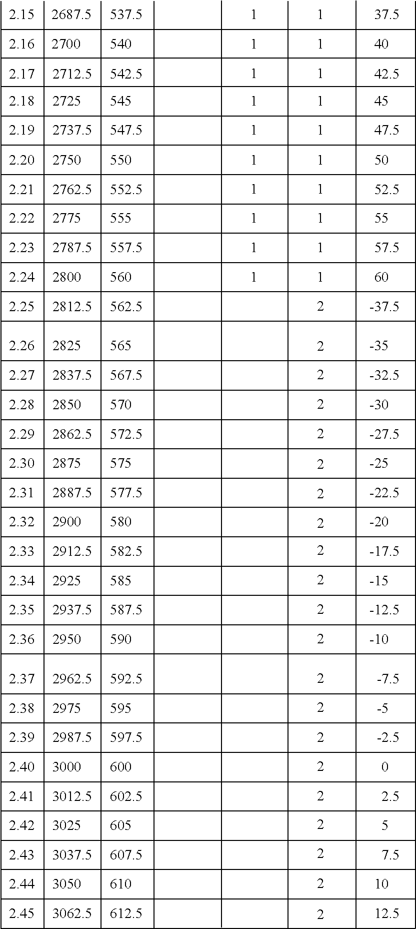

Table 8 shows the required dosage in milligrams, the required dosage volume in mL, the number of various IV bags required and the top-up/surplus volume for a dose of 750 mg of gemcitabine per m² of BSA.

TABLE 8
gemcitabine dose reduction:

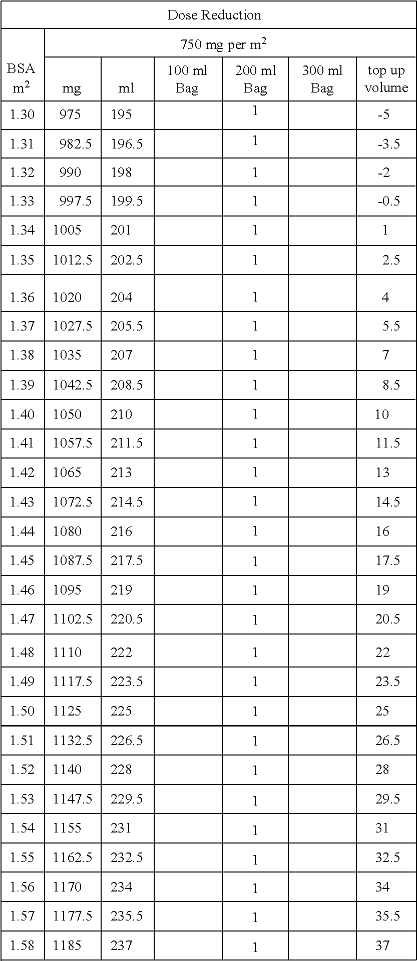

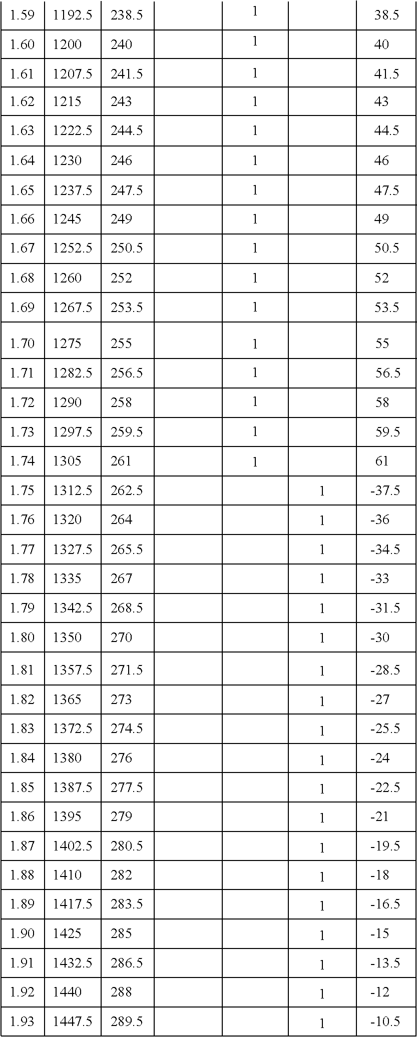

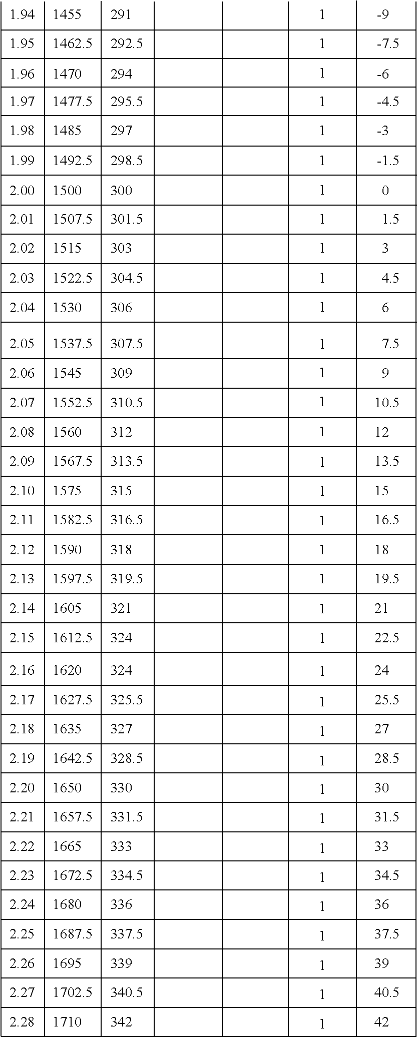

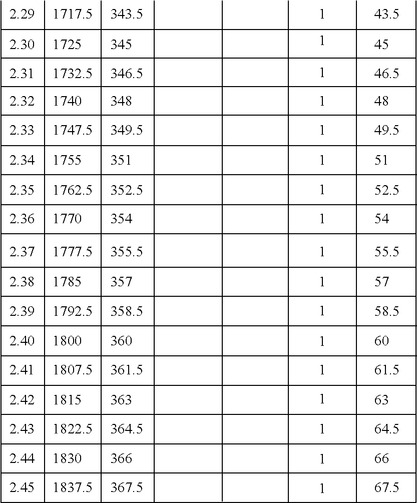

1. A method for intravenous delivery of a patient-adjusted dose of a therapeutic agent in a solution of predetermined concentration to a patient in need thereof, comprising:
administering intravenously to the patient in need thereof a dosage volume of the solution having a predetermined concentration of the therapeutic agent,
wherein the predetermined concentration of the therapeutic agent is used for each patient treated with the therapeutic agent at a particular dosage strength, and
wherein the predetermined concentration of the therapeutic agent is independent of the patient-adjusted dose,
and wherein the dosage volume is selected to deliver the patient-adjusted dose to the patient.

2. The method of claim 1, wherein the patient-adjusted dose of the therapeutic agent is a function of at least one patient characteristic.

3. The method of claim 2, wherein the patient characteristic selected from the group consisting of one of the patient's body surface area, the patient's body weight or ideal body weight, the patient's hepatic function, and the patient's renal function.

4. The method of claim 1, wherein the therapeutic agent is selected from the group consisting of an antibiotic, an antiviral, a muscle relaxant, a sedative, an anesthetic, a cancer chemotherapeutic agent, and an antifungal.

5. The method of claim 1, wherein the therapeutic agent is selected from the group consisting of irinotecan, doxorubicin, cisplatin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine.

6. The method of claim 1, wherein the therapeutic agent is a glucocorticoid.

7. The method of claim 1, wherein the therapeutic agent is selected from the group consisting of dexamethasone and cortisol.

8. The method of claim 1, wherein the therapeutic agent comprises irinotecan.

9-15. (canceled)

16. A system for intravenous delivery of a patient-adjusted dose of a therapeutic agent in a solution of predetermined concentration to a patient in need thereof of claim 1, comprising:
at least one pharmacy container containing the solution of predetermined concentration of the therapeutic agent, wherein the at least one pharmacy container is configured to allow transfer of multiple portions of the solution;
one or more patient dosing containers each containing an initial volume of the solution of predetermined concentration of the therapeutic agent, wherein the one or more patient dosing containers are configured to allow volume adjustment and are configured for intravenous administration of the solution to the patient,
wherein each of the one or more patient dosing containers may contain the same or different initial volumes of the solution, and
wherein the predetermined concentration of the solution in the at least one pharmacy container and the one or more patient dosing containers is the same concentration.

17. The system of claim 16, wherein the volume adjustment comprises introduction of a top-up volume of the solution from the at least one pharmacy container into one or more of the one or more patient dosing containers.

18. The system of claim 16, wherein the volume adjustment comprises withdrawal of a surplus volume of the solution from one or more of the patient dosing containers.

19. The system of claim 16, wherein the volume adjustment comprises introduction of a top-up volume of the solution from the at least one pharmacy container into at least one patient dosing container, and withdrawal of a surplus volume of the solution from at least one other patient dosing container.

20. The system of claim 16, further comprising instructions for preparing the patient-adjusted dose of the therapeutic agent, wherein the instructions indicate that the patient-adjusted dose of the therapeutic agent can be prepared using a specified volume of the solution of predetermined concentration.

21-22. (canceled)

23. The system of claim 16, wherein the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume.

24. The system of claim 23, further comprising instructions for preparing the patient adjusted dose, wherein the instructions describe the number of patient dosing containers from the first group and the number of patient dosing containers from the second group and the volume, if any, of the solution of predetermined concentration from the pharmacy container required to provide the patient-adjusted dose.

25-34. (canceled)

35. The system of claim 16, wherein the therapeutic agent is selected from the group consisting of an antibiotic, an antiviral, a muscle relaxant, a sedative, an anesthetic, a cancer chemotherapeutic agent, and an antifungal.

36. The system of claim 16, wherein the therapeutic agent is selected from the group consisting of irinotecan, doxorubicin, cisplatin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine.

37. The system of claim 16, wherein the therapeutic agent is a glucocorticoid.

38. The system of claim 16, wherein the therapeutic agent is selected from the group consisting of dexamethasone and cortisol.

39. The system of claim 16, wherein the therapeutic agent comprises irinotecan.

40. (canceled)

41. The system of claim 16, wherein the therapeutic agent comprises irinotecan, and wherein the predetermined concentration is selected from the group consisting of 0.72 mg/mL, 0.6 mg/mL, 0.48 mg/mL, 0.6-0.8 mg/mL, 0.5-0.7 mg/mL, and 0.4-0.6 mg/mL.

42-47. (canceled)

48. A method of preparing a patient-adjusted dose of a therapeutic agent for intravenous delivery to a patient in need thereof using the system of claim 16, comprising:
determining a dosage volume of the solution of predetermined concentration of the therapeutic agent to be administered to the patient, wherein the dosage volume is the volume of solution required to provide the patient-adjusted dose; and
selecting one or more patient dosing containers containing the solution of predetermined concentration of the therapeutic agent, wherein
if the total volume in the one or more patient dosing containers is less than the dosage volume, then adding a top-up volume of the solution of predetermined concentration of the therapeutic agent from a pharmacy container to one or more of the one or more patient dosing containers, such that after addition of the top-up volume, the total volume of solution in the one or more patient dosing containers is equal to the dosage volume, wherein the predetermined concentration of the solution in the pharmacy container and the one or more patient dosing containers is the same concentration, and wherein the pharmacy container is configured to allow transfer of multiple portions of the solution,
or
if the total volume in the one or more patient dosing containers is more than the dosage volume, then withdrawing a surplus volume of the solution of predetermined concentration from one or more of the one or more patient dosing containers, such that after withdrawal of the surplus volume, the total volume of solution in the one or more patient dosing containers is equal to the dosage volume,
wherein the one or more patient dosing containers are configured to allow volume adjustment and are configured for intravenous administration of the solution to the patient.

49. The method of claim 48, wherein the total volume in the one or more patient dosing containers is less than the dosage volume.

50. The method of claim 49, wherein volume adjustment comprises introduction of a top-up volume of the solution from at least one pharmacy container into one or more of the one or more patient dosing containers.

51. The method of claim 48, wherein the total volume in the one or more patient dosing containers is more than the dosage volume.

52. The method of claim 51, wherein volume adjustment comprises withdrawal of a surplus volume of the solution from one or more of the patient dosing containers.

53. The method of claim 48, wherein the volume adjustment comprises introduction of a top-up volume of the solution from the at least one pharmacy container into at least one patient dosing container, and withdrawal of a surplus volume of the solution from at least one other patient dosing container.

54-55. (canceled)

56. The method of claim 48, wherein the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume.

57-64. (canceled)

65. The method of claim 48, wherein the therapeutic agent is selected from the group consisting of an antibiotic, an antiviral, a muscle relaxant, a sedative, an anesthetic, a cancer chemotherapeutic agent, and an antifungal.

66. The method of claim 48, wherein the therapeutic agent is selected from the group consisting of irinotecan, doxorubicin, cisplatin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine.

67. The method of claim 48, wherein the therapeutic agent is a glucocorticoid.

68. The method of claim 48, wherein the therapeutic agent is selected from the group consisting of dexamethasone and cortisol.

69. The method of claim 48, wherein the therapeutic agent comprises irinotecan.

70-77. (canceled)

78. A method of manufacturing the system of claim 48 for intravenous delivery of a patient-adjusted dose of a therapeutic agent in a solution of predetermined concentration to a patient in need thereof, comprising:
formulating a solution for intravenous delivery, wherein the solution comprises a predetermined concentration of the therapeutic agent; and
packaging the solution in a plurality of containers, wherein the plurality of containers comprise one or more patient dosing containers and at least one pharmacy container, wherein the one or more patient dosing containers are configured to allow volume adjustment and are configured for intravenous administration of the solution to a patient, and wherein the at least one pharmacy container is configured to allow transfer of multiple portions of the solution.

79. The method of claim 78, wherein volume adjustment comprises introduction of a top-up volume of the solution from at least one pharmacy container into one or more of the one or more patient dosing containers.

80. The method of claim 78, wherein the volume adjustment comprises withdrawal of a surplus volume of the solution from one or more of the patient dosing containers.

81. The method of claim 78, where the volume adjustment comprises introduction of a top-up volume of the solution from the at least one pharmacy container into at least one patient dosing container, and withdrawal of a surplus volume of the solution from at least one other patient dosing container.

82-83. (canceled)

84. The method of claim 78, wherein the one or more patient dosing containers comprise a first group of one or more patient dosing containers and a second group of one or more patient dosing containers, wherein each of the one or more patient dosing containers in the first group contains a first volume of the solution of predetermined concentration and each of the one or more patient dosing containers in the second group comprises a second volume of the solution of predetermined concentration, and wherein the first volume is different from the second volume.

85-92. (canceled)

93. The method of claim 78, wherein the therapeutic agent is selected from the group consisting of an antibiotic, an antiviral, a muscle relaxant, a sedative, an anesthetic, a cancer chemotherapeutic agent, and an antifungal.

94. The method of any claim 78, wherein the therapeutic agent is selected from the group consisting of irinotecan, doxorubicin, cisplatin, leukovorin, 5-fluorouracil, paclitaxel, docetaxel, carboplatin, oxaliplatin, gemcitabine, navelbine, vincristine sulphate, ifosfamide, cyclophosphamide, daunorubicin, epirubicin, idarubicin, mechlorethamine, mitomycin, mitoxantrone, streptozocin, teniposide, vinblastine, vincristine, vinorelbine, vancomycin, methicillin, acyclovir, amikacin, atracurium, daptomycin, gentamicin, midazolam, propofol, remifentanil, rocuronium, tobramycin, vecuronium, voriconazole, carmustine, cladribine, dacarbazine, dolesatrone, fulvestrant, pamidronate, pegfilgrastim, plicamycin, and vinorelbine.

95. The method of any claim 78, wherein the therapeutic agent is a glucocorticoid.

96. The method of claim 78, wherein the therapeutic agent is selected from the group consisting of dexamethasone and cortisol.

97. The method of claim 78, wherein the therapeutic agent comprises irinotecan.

98-105. (canceled)

106. The method of claim 78, wherein packaging the solution in the plurality of containers comprises aseptic filtration of the solution.
